(19) United States
(12) Patent Application Publication
(10) Pub. No.: US 2004/0087485 A1
(43) Pub. Date: May 6, 2004

(54) Educated nkt cells and their uses in the treatment of immune-related disorders

(76) Inventors: Yaron Iian, Givat Massua (IL); Maya Margalit, Givat Mordechai (IL); Eran Elinav, Ramat Beit Hakerem (IL)

Correspondence Address: Natalie Bogdanos, Associate Corporation & Patent Counsel, Enzo Biochem Inc 9th Floor, 527 Madison Avenue, New York, NY, 10022-4304, US

(21) Appl. No.: 10/451,811

(22) PCT Filed: Dec. 24, 2001
(86) PCT No.: PCT/IL01/01197

(30) Foreign Application Priority Data
Dec. 25, 2000 (IL) 140537

(51) Int. Cl. A61K 38/16

(52) U.S. Cl. 514/2

(57) ABSTRACT

The present invention relates to a method for the treatment of immune-related disorders in a mammalian subject in need of such treatment. This method comprises the step of manipulating the NK T cell population in said subject by suitable means, said manipulation of the NK T cell population resulting in modulation of the Th1/Th2 balance toward anti-inflammatory cytokine producing cells. Manipulation of the NK T cell population may be performed either by depletion of said cells by a suitable means or alternatively by ex vivo education of the NK T cells, such that the educated NK T cells have the capability to modulate the Th1/Th2 balance toward anti-inflammatory cytokine producing cells.

The invention further relates to pharmaceutical compositions for the treatment of immune-related disorders in a mammalian subject. These compositions comprising as an effective ingredient an ex vivo educated NK T cell. The invention further provides for an ex vivo educated NK T cell and uses thereof in the treatment of immune-related disorders.

### FIELD OF THE INVENTION

The present invention relates to the field of therapeutic methods, compositions and uses thereof, in the treatment of immune-related disorders in mammalian subjects. More particularly, the methods and the compositions of the invention are directed to manipulation of the NK T cell population in a subject, that results in modulation of the Th1/Th2 cell balance toward anti-inflammatory cytokine producing cells, and to their use in the treatment of intestinal and immune related disorders and of malignancies.

All patents, patent applications, patent publications, scientific articles and the like, cited or identified in this application are hereby incorporated by reference in their entirety in order to describe more fully the state of the art to which the present application pertains.

### BACKGROUND OF THE INVENTION

The immune system is responsible for a major part of the defense against potentially harmful agents. However, this system may turn against self antigens, and bring about autoimmune disorders such as inflammatory bowel disease. These disorders can be perceived as a dysbalance between pro-inflammatory (Th1) and anti-inflammatory (Th2) cytokines.

Overcoming the immune response tends to involve generalized immunosuppression which can often lead to undesirable side effects. Thus, there is a need for an alternative strategy for induction of antigen-specific immune suppression. Immune tolerance can be induced by two types of mechanisms. The first, termed “recessive”, involves clonal anergy or deletion of the vast majority of the immunocytes that are capable of responding to the antigen [Matzinger, P. et al., Ann. Rev. Immunol. 12:991-1045 (1994); Qin, S., et al., Science 259:974-977 (1993)]. Alternatively, in a “dominant” type of tolerance, negative immunoregulatory lymphocytes may emerge as a result of tolerization procedures. In contrast to clonal deletion or anergy, the presence of a limited number of these lymphocytes may down regulate a much larger number of effector cells.

The Role of the Immune System in the Pathogenesis of Inflammatory Bowel Disease

Inflammatory bowel diseases (IBD) are common gastrointestinal disorders, that can be perceived as being the result of a dysbalance between Th1-pro-inflammatory, and Th2-anti-inflammatory subtypes of immune responses [Strober, W., et al., Immunol Today 18:61-64 (1997); Neurath, M., et al., J. Exp. Med. 183:2605-2616 (1996)].

There are several extra-intestinal manifestations that accompany IBD, for example: autoimmune phenomena; immune complexes have a role in target organ damage; and, immunosuppressive agents such as glucocorticoids, azathioprine, methotrexate and cyclosporin are used to alleviate the disease [Podolsky, D. K., et al., New Engl. J. Med., 325:928-935(1991); Strober, W., et al., In Clinical Immunology, Mosby, St. Louis. R. R. Rich, Editor, 1401-14281-2 (1995)]. Patients with IBD have antibodies against components of colon cells and several different bacterial antigens. These antigens gain access to the immune system as a consequence of epithelial damage [Hibi, S., et al., Clin. Exp. Immunol. 54:163-168 (1983); Das, K. M., et al., Gastroenterology 98:464-69 (1990)]. Abnormalities of T cell-mediated immunity, including coetaneous anergy and diminished responsiveness to T cell stimuli, have also been described in these patients [Chiba, M., et al. Gut, 22:177-182 (1981); Raedler, A., et al., Clin. Exp. Immunol. 60:518-526 (1985)]. In addition, changes in mucosal cell mediated immunity were identified, including increased concentrations of mucosal IgG cells and changes in T cells subsets, suggesting antigen stimulation [Dasgupta, A., et al., Gut 35:1712-17 (1994); Takahashi, F., et al., J. Clin. Invest. 76:311-318 (1985)]. Exposure of target antigens after infectious, immune, or toxic damage, leads to activation of mucosal immune cells resulting in cytokines that lead to mucosal inflammatory response [Neurath, M., et al., J. Exp. Med., 183:2605-2616 (1996)]. Secretion of pro-inflammatory cytokines such as IFNγ, contributes to an increase in mucosal permeability, and has been described in animal models of IBD [Strober, W., et al., Immunol. Today 18:61-64. (1997)]. Similarly, an increase in collagen synthesis mediated by IL1 and IL6 can be detected in these animals [Strober, W., et al., ibid.]. A Th1-mediated granulomatous colitis model has been established by the adoptive transfer of normal CD45RB T cells from Balb/C mice into CB-17 scid mice. CD4 cells from CD45RB were shown to prevent the disease when injected together with the CD45RB population. This prevention could be reversed by adding antibodies to TGFβ1 [Sadlack, B., et al., Cell 75:253-261 (1993); Powrie, F., et al., Immunity 1:553-562 (1994)].

The Th1/Th2 Dysbalance in Inflammatory Bowel Disease

Both CD4 and CD8 lymphocytes can be typed as either Th1 cells that produce IL-2 and IFNγ, or Th2 cells that produce IL-4, and IL-10. The way the immune system responds to foreign and self antigens, is the result of a balance between the two subtypes of responses [Weiner, H. L., et al., Immunol. Today 18: 335-343 (1997); Adorini, L., et al., Immunol. Today 18:209-211 (1997); Rabbani, E. et al., European Patent Publication No. EP1149586A1 (filed on Apr. 27, 2001), herein incorporated by reference]. A Th1 type response is involved in the pathogenesis of several autoimmune and chronic inflammatory disorders such as IBD [Adorini, L., et al., (1997) ibid.; Mizoguchi, A., et al., J. Exp. Med. 183:847-856, (1996)]. Thus experimental colitis and IBD in humans can be perceived as a dysbalance between pro-inflammatory Th1-type and anti-inflammatory Th2-type cytokines. It has been recently shown, in both animals and humans, that anti-inflammatory cytokines such as IL10 can downregulate the pro-inflammatory effects of Th1-mediated cytokines, thereby alleviating immune-mediated disorders [Mizoguchi, A., et al., (1996) ibid.; Madsen, K. L., et al., Gastroenterology 113:151-159 (1997); Van Deventer Sander, J., et al., Gastroenterology 113:383-389 (1997)].

Oral Tolerance Induction for Amelioration of Immune-mediated Disorders

Oral tolerance is a recognized procedure for the induction of antigen-specific peripheral immune hyporesponsiveness [Weiner, H. L., et al., (1997) ibid.; Weiner, H., Proc. Natl. Acad. Sci. USA 91:10762-10765 (1994); Roy-Chowdury, et al., International Publication No. WO 98/37917 (filed Feb. 26, 1998), herein incorporated by reference]. Oral administration of antigens has been shown, both in animals and humans, to prevent or alleviate several autoimmune disorders such as collagen-induced arthritis, uveitis, diabetes, and experimental allergic encephalomyelitis [Esbjorn, T., et al., Int. Arch. Allergy Immunol. 113:219-223 (1997);, Von Herrath, M. G., et al., J. Clin. Inves. 98:1324-1331 (1996); Hancock, W., et al., Am. J. Path. 147:1193-1197 (1993); Weiner, H. L., et al., Science 261:1321-1324 (1993)].

Enteral exposure to high doses of the antigens induces tolerance by clonal inactivation of antigen specific T cells, while the feeding of low doses of the antigens leads to induction of regulatory cell secreting factors that suppress the generation of antigen-specific effector cells [Weiner, H. L., et al., (1997) ibid.]. Both in animals and humans, tolerance induction is associated with a Th2/Th3 type immune response leading to the secretion of immunosuppressive cytokines such as IL10, IL4 and TGFβ1 [Weiner, H. L., et al., (1997) ibid.]. A bystander effect involving reactivity to multiple closely-related-antigens, was shown to play a role in oral tolerance induction in several models [Weiner, H. L., et al., (1997) ibid.; Carvalho, B. A., et al., Scand J. Immunol. 45: 276-281(1997)]. As regulatory cells secrete non-antigen specific cytokines after being triggered by a fed antigen, they can suppress inflammation in the microenvironment where the fed antigen is localized. Although the procedure is well established as a method for immune tolerance induction, the exact mechanism has yet to be discovered. Conflicting results have been published as to whether an antigen has to be processed and/or absorbed, and whether protein denaturation is necessary for tolerance induction [Carvalho, B. A., et al., (1997) ibid.; Blanas, E., et al., Science 274:1707-1709 (1996)].

Antigen presentation may require whole proteins to be presented into the bowel, however protein processing and absorption may also be involved in tolerance induction or in its maintenance through post-gut mechanisms [Carvalho, B. A., et al., (1997) ibid.]. Gut wall epithelial cells, Peyer's patches, mesenteric lymph node, or extraintestinal cells have been suggested as mediating immune tolerance induction [Strober, W., et al., (1997) ibid.]. However, oral administration of an antigen can also elicit an epitope-specific immunity [Carvalho., B. A., et al., (1997) ibid.; Blanas, E., et al (1996) ibid.]. Indeed, side by side with immunosuppressive-cytokines-secreting cells (e.g. Th3 cells secreting TGFβ) that appear after oral tolerization, a second population of cells, secreting pro-inflammatory-cytokines (e.g. IFNγ,) can be found in the gut wall, mainly in Peyer's patches [Weiner, H. L., et al., (1997) ibid.]. Orally administered antigen elicits a local pro-inflammatory response, IFNγ-mediated, in the gut mucosa, along with a systemic TGFβ and IL4-mediated anti-inflammatory response. In contrast to splenocytes from orally tolerized animals, gut extracted lymphocytes have been unable to transfer the tolerance into naive animals [Strober, W., et al., (1997) ibid.; Weiner, H. L,. et al., (1997) ibid.]. Thus induction of oral tolerance requires a balance between an immunogenic and a tolerogenic cell population, with a shift from a Th1 (and secretion of pro-inflammatory cytokines), to a Th2 (and secretion of anti-inflammatory cytokines) immune response.

It has been shown by others and the present inventors that oral tolerance can be used to prevent or alleviate experimental colitis in a model system that employs mice treated with 2,4,6-trinitrobenzenesulfonic acid (TNBS) [Madsen, K. L., et al., Gastroenterology 113:151-159 (1997); Trop, S., et al., Hepatology 27:746-755 (1999)]. Induction of oral tolerance to colitis extracted proteins downregulates the anti-colon immune response, thereby ameliorating the immune-mediated-colitis. Suppressor lymphocytes mediate the tolerance by induction of a shift from a pro-inflammatory to an anti-inflammatory immune response [Madsen, K. L., et al., (1997) ibid.; Trop, S., et al., ibid.].

The Role of the Liver in Immune Tolerance Induction

The liver has long been suggested to be involved in immunoregulatory functions. It is the largest reticuloendothelial organ in the body, and several subpopulations of its cells are involved in antigen presentation and/or processing [Callery, M. P., et al., J. Surg. Res. 46:391-394 (1989); Nakano, Y., et al., Surgery 111:668-676 (1992); Yu, S. Y., et al., Surgery 116:229-234 (1994)].

Portocaval shunts, or blockage of Kupffer cell functions have precluded induction of oral tolerance in several animal models [Callery, M. P., et al., (1989) ibid.; Nakano, Y., et al., (1992) ibid.; Yu, S. Y., et al., (1994) ibid.].

Antibody titers to intestinal flora were found to be elevated in humans with chronic liver diseases that underwent portocaval shunts [Crispe, N., et al., Immun. Today 11:236-245 (1996); Ilan, Y., et al., Gastro 114:260 (1998)].

Portal vein administration of donor cells has been shown to promote allo-specific hyporesponsiveness [Crispe, N., et al., (1996) ibid.]. Thus, the liver may be necessary for peripheral immune tolerance induction through first pass clearance of specific subpopulations of cells or peptides.

Liver-associated Lymphocytes

The adult liver contains several subpopulations of cells involved in its immunomodulatory functions. Kupffer cells were found important in front line defense against antigens entering the liver through portal circulation. Antigen-activated Kupffer cells have antigen presentation, phagocytosis, and have exhibited killing properties via secretion of cytokines. These cells also induce chemotaxis and lymphocyte aggregation [Crispe, N., et al., (1996) ibid.]. In addition, the adult liver contains pluripotent stem cells, giving rise to multiple cell lineages including thymic and extrathymic T cells, granulocytes, and erythroid lineage cells [Crispe N, et al., (1996) ibid.]. Indeed, T cells can differentiate extrathymically in an adult liver [Collins C., et al., Eur. J. Immunol. 26:3114-3118 (1996)].

The liver appears to be the meeting place for two populations of T cells consisting of thymus derived T cells with high TCR (TCR^{high}) and extrathymic T cells with intermediate TCR (TCR^{int}). The first set of T cells, also known as mainstream T cells, contains a mixture of minor populations of CD4⁺ and CD8⁺ cells, and a large population of CD⁴⁻⁻CD8⁻⁻ double negative (DN) cells, that do not express NK cell markers or IL2Rβ, and which are closely linked to the circulating T cells pool [Crispe, N., et al., (1996) ibid.]. Many of the DN cells express a B cell marker, B220, induction of which leads to trafficking of apoptosing T cells to the liver [Crispe, N., et al., (1996) ibid.; Ilan, Y., et al., (1998) ibid.; Collins, C., et al., (1996) ibid.; Garcia-Barcina, M., et al., Immunol 82:95-8 (1994); MacDonald, R. H., et al., J. Exp. Med. 182:633-638 (1995)]. The second subset of liver T cells, known as alternative T cells, are CD4⁺, or CD4-8⁻ and CD16⁻, express αβTCR^{int}, and known NK receptors including NKR-P1, Ly-49A, and IL2 receptor β-chain [Garcia-Barcina, M., et al., (1994) ibid., MacDonald, R. H., et al., (1995) ibid.; Bendelac, A., et al., Curr. Opin. in Immunol. 7:367-374. (1995)]. The majority of the liver IL2Rβ⁺ TCR^{int} cells are NK1.1⁺. These cells are rare in the pool that circulates through the peripheral lymphoid organs. A small population of these cells, however, is present in the thymic medulla, spleen, and bone marrow. TCR^{int} IL2Rβ⁺ NK1.1+ cells differentiate through primordial pathway, thymic and extrathymic alternative pathways, rather than through the conventional thymic pathway, and can develop in livers of thymectomized animals [MacDonald, R. H., et al., (1995) ibid.; Bendelac, A., et al., (1995) ibid.; Takahashi, M., et al., J. Immunol. 156: 2436-2442 (1996); Doherty, D. G., et al., Hepatology 26:445A (1997)]. Their functions are not characteristic of those of any subset of conventional T cells, but include elements of cytotoxicity and B cell help. Upon primary activation they release a large variety of cytokines of both Th1 and Th2 origin [MacDonald, R. H., et al., (1995) ibid.; Bendelac, A., et al., (1995) ibid.; Takahashi, M., et al., (1996) ibid.; Doherty, D. G., et al., (1997) ibid.]. They also respond to IL12 and produce IFNγ, both of which are Th1 cytokines, inducing anti-tumor and anti-microbial effector cells [Takahashi, M., et al., (1996) ibid.; Doherty, D. G., et al., (1997) ibid.]. In addition, in the liver these cells multiply in response to IL12 and TNFα, and may be actively involved in lethal hit to mainstream T cells during peripheral deletion [Takahashi, M., et al., (1996) ibid.; Doherty, D. G., et al., (1997) ibid.].

One of the objects of the present invention is to determine the role of NK1.1⁺ lymphocytes in peripheral immune tolerance induction, in induction of tolerance and/or inflammation via adoptive transfer of splenocytes, specifically in keeping the balance between immunogenic and tolerogenic subsets of lymphocytes. The results of the present study show for the first time, that NK1.1⁺ lymphocytes may play a dual role in immune mediated disorders. In a “tolerized environment”, they induce and/or maintain immune hyporesponsiveness via alteration of the Th1/Th2 paradigm in the anti-inflammatory direction. On the other hand, in a “non-tolerized environment”, they support a pro-inflammatory paradigm. This and other objects of the invention will become clearer as the description proceeds.

### Summary of the Invention

In a first aspect, the invention relates to a method for the treatment of immune-related disorders in a mammalian subject in need of such treatment, by manipulating the NK T cell population in said subject by suitable means, said manipulation of the NK T cell population resulting in modulation of the Th1/Th2 cell balance toward anti-inflammatory cytokine producing cells.

In a preferred embodiment, the invention relates to method of manipulating the NK T cell population by depletion of said cells. As specifically preferred embodiment, depletion of the NK T cell population may be performed by administering to the subject a therapeutically effective amount of a composition comprising as the effective ingredient an antibody that specifically recognizes the NK T cells. Alternatively, depletion of the NK T cell population may be performed by ex vivo pheresis, using beads coated with an antibody that specifically recognizes the NK T cells.

In an alternatively preferred embodiment, the invention relates to a method for the treatment of immune-related disorders in a mammalian subject, this method involving manipulation of NK T cell population by ex vivo education of the said NK T cells, such that the educated NK T cells have the capability to modulate the Th1/Th2 balance toward anti-inflammatory cytokine producing cells.

A specifically preferred embodiment relates to a method for treatment of immune-related disorders in a mammalian subject, comprising the steps of:

a. obtaining NK T cells from said subject;

b. ex vivo educating the NK T cells obtained in step (a) such that the resulting educated NK T cells have the capability of modulating the Th1/Th2 cell balance toward anti-inflammatory cytokine producing cells; and

c. re-introducing to the subject the educated NK T cells that were obtained in step (b). Modulation of the Th1/Th2 cell balance toward anti-inflammatory cytokine producing cells, results in an increase of the quantitative ratio between any one of IL4 and IL10 to IFNγ.

More specifically, ex vivo education of the NK T cells may be performed by culturing these cells in the presence of any one of:

a. antigens associated with the immune-related disorder to be treated or any combination thereof;

b. at least one liver-associated cells of tolerized or non-tolerized patients suffering from the same immune-related disorder or from said subject;

c. at least one cytokines or adhesion molecules; and

d. a combination of any of (a), (b) and (c) above.

The method of the invention may optionally further comprise the step of eliciting in the subject up or down regulation of the immune response to the immune-related disorder, preferably by oral tolerization.

The ex vivo educated NK T cells according to the method of the invention are re-introduced by adoptive transfer to the treated subject.

Another preferred embodiment relates to the method of the invention wherein the immune-related disorder is an inflammatory bowel disease (IBD). More particularly, said disease may be Crohn's disease.

In another specifically preferred embodiment, the method of the invention is intended for the treatment of a malignancy selected from the group consisting of melanomas, carcinomas, lymphomas and sarcomas. For this purpose, NK T cells may be manipulated in the direction of enhancing the immune response in a pro-inflammatory direction, in order to augment the favorable anti-tumor immunity.

In yet another specifically preferred embodiment, the method of the invention is intended for the treatment of human patients.

As second aspect the present invention relates to a therapeutic composition for the treatment of immune-related disorder in a mammalian subject. The composition of the invention comprises as an effective ingredient ex vivo educated autologous NK T cells capable of modulating the Th1/Th2 cell balance toward anti-inflammatory cytokine producing cells. These educated autologous NK T cells mediate increase in the quantitative ratio between any one of IL4 and IL10 to IFNγ. The composition of the invention may optionally further comprise pharmaceutically acceptable carrier, dilluent, excipient and/or additive.

In a preferred embodiment, the educated autologous NK T cell comprised in the therapeutic composition of the invention is cultured ex vivo, prior to its use in the composition of the invention, in the presence of any one of:

a. antigens associated with said immune-related disorder or any combination thereof,

b. at least one of liver-associated cells of tolerized or non-tolerized patients suffering from said immune-related disorder or from the subject to be treated;

c. at least one of cytokines, or adhesion molecules; and

d. a combination of any of (a), (b) and (c) above;

In one preferred embodiment the therapeutic composition of the invention is intended for the treatment of intestinal inflammatory disease in a mammalian subject, particularly humans, and more specifically for the treatment of Crohn's disease.

In another preferred embodiment the therapeutic composition of the invention is intended for the treatment of a malignancy selected from the group consisting of melanomas, carcinomas, lymphomas and sarcomas.

In yet another preferred embodiment the invention relates to a therapeutic composition for the treatment of immune-related disorder. This composition comprising as an effective ingredient an antibody that specifically recognizes the NK T cells.

In one embodiment, the therapeutic composition of the invention may be used for the treatment of an intestinal inflammatory disease, such as Crohn's disease.

In another embodiment the therapeutic composition of the invention may be used for the treatment of a malignancy selected from the group consisting of melanomas, carcinomas, lymphomas and sarcomas. For this purpose, NK T cells may be manipulated in the direction of enhancing the immune response in a pro-inflammatory direction, in order to augment the favorable anti-tumor immunity.

As a third aspect, the present invention relates to the use of educated autologous NK T cells in the manufacture of a therapeutic composition for modulating the Th1/Th2 balance toward anti-inflammatory cytokine producing cells, in a mammalian subject suffering of a immune-related disorder.

In a specifically preferred embodiment, the invention relates to the use of ex vivo educated autologous NK T cells in the manufacture of a therapeutic composition for the treatment of immune-related disorder in a mammalian subject. The educated autologous NK T cells are capable of modulating the Th1/Th2 cell balance toward anti-inflammatory cytokine producing cells, and thus mediate an increase in the quantitative ratio between any one of IL4 and IL10 to IFNγ.

In one specifically preferred embodiment the invention relates to use of ex vivo educated autologous NK T cells, in the manufacture of a therapeutic composition for the treatment of intestinal inflammatory disease in a mammalian subject, particularly human patients and especially for the treatment of Crohn's disease

In another specifically preferred embodiment the invention relates to use of ex vivo educated autologous NK T cells, in the manufacture of a therapeutic composition for the treatment of a malignancy, more specifically a for the treatment of melanomas, carcinomas, lymphomas and sarcomas.

The present invention further provides for an ex vivo educated autologous NK T cell for use in the treatment of immune-related disorders in a mammalian subject in need of such treatment. The educated NK T cell has been ex vivo cultured in the presence of any one of:

a. antigens associated with said immune-related disorder or any combination therof;

b. at least one of liver-associated cells of tolerized or non-tolerized patients suffering from said immune-related disorder or of said subject;

c. at least one of cytokines, or adhesion molecules; and

d. a combination of any of (a), (b) and (c) above.

In another embodiment of the present aspect, the invention relates to the use of an ex vivo educated autologous NK T cell in the treatment of immune-related disorders in a mammalian subject in need of such treatment.

In yet another preferred embodiment the present invention relates to the use of an antibody that specifically recognizes NK T cells, in the manufacture of a therapeutic composition for manipulation of the NK T cells population in a mammalian subject suffering of a immune-related disorder, more specifically, the depletion of said NK T cell population.

The depletion of the NK T cells population results in modulating the Th1/Th2 cell balance toward anti-inflammatory cytokine producing cells.

In a specifically preferred embodiment, the invention relates to the use of an antibody that specifically recognizes the NK T cells, in the manufacture of a therapeutic composition for the treatment of immune-related disorder in a mammalian subject.

In one specific embodiment the immune related disorder may be an intestinal inflammatory disease, such as Crohn's disease. In another specific embodiment, the immune-related disorder may be a malignancy, such as melanoma, carcinoma, lymphoma and sarcoma.

### BRIEF DESCRIPTION OF THE FIGURES

FIGS. 1A-1B: Effect of tolerization on histologic evaluation of bowel mucosa in experimental colitis

FIG. 1A: shows paraffin sections from distal colonic tissue (last 10 cm) of non-tolerized mice.

FIG. 1B: shows paraffin sections from distal colonic tissue (last 10 cm) of tolerized mice.

Sections were stained with hematoxylin-eosin. Feeding of mouse-derived CEP led to marked alleviation of experimental colitis, manifested by marked reduction in inflammatory response and mucosal damage (group B, FIG. 1B). In contrast, severe colitis was observed in non-tolerized mice fed with BSA (group A, FIG. 1A).

FIG. 2: NK1.1+ lymphocytes increase the CD4+IL4+/CD4+IFNγ+ ratio in tolerized mice

Splenocytes and liver-associated-lymphocytes (LAL) (2.5×10⁶ splenocytes and 0.5×10⁶ LAL) were harvested from mice in all groups and cultured for 72 hours in the presence of CEP and APC. Flow cytometry analysis summarized in the following histogram has shown that NK1.1-LAL depletion following oral tolerance induction decreased the CD4+IL4+/CD4+IFNγ+ ratio (group B, black bar) in comparison with the non-NK1.1 depleted tolerized mice (group E, white bar). Control NK1.1-depleted group (group F, black bar) had a decrease in CD4+IL4+/CD4+IFNγ+ ratio compared with non-NK1.1-depleted group (group C, white bar). Abbreviations: EXP. GR.=Experimental groups, rat.=ratio, CEP=colitis extracted protein, n-dep.=none-depleted, NK1.1-dep. (NK1.1-depleted), cont.=control.

FIG. 3: NK1.1+ lymphocytes decreased the CD4+IL4+/CD4+IFNγ+ ratio in non-tolerized mice with experimental colitis

In contrast to tolerized groups, NK1.1-depletion had an opposite effect in non-tolerized mice with experimental colitis (N-CEP). The CD4+IL4+/CD4+IFNγ+ ratio increased in NK1.1-depleted non tolerized group (group D, black bar), in comparison with the non-NK1.1 depleted non-tolerized group (groups A, white bar). Abbreviations: EXP. GR.=Experimental groups, rat.=ratio, CEP=colitis extracted protein, n-dep.=none-depleted, NK1.1-dep. (NK1.1-depleted), cont.=control.

FIG. 4: Expression of IL4 and IFNγ on isolated lymphocytes from different experimental groups

The figure shows representative results of flow cytometry analysis for determination of IL4 and IFNγ expression. Expression of IL4 and IFNγ in isolated lymphocytes from tolerized NK1.1 non-depleted and depleted mice from groups B and E, and non-tolerized NK1.1 non-depleted and depleted mice from groups A and D, respectively. Data are displayed as dot plots after gating of 5×10⁴ small lymphocytes. Numbers below dot plots represent the percentages of stained cells.

The different experimental groups are indicated by B, E, A and D. Abbreviations: EXP. GR.=Experimental groups.

FIG. 5: The effect of in-vitro antigen exposure on CD4+IL4+/CD4+IFNγ ratio in tolerized and non-tolerized mice with experimental colitis

For evaluation of the effect of disease-target antigen on the CD4+IL4+/CD4+IFNγ+ ratio, splenocytes and liver-associated-lymphocytes (2.5×10⁶ splenocytes and 0.5×10⁶ LAL) were harvested from mice of all groups (B, E, A, D, C, F) and cultured for 12 hours in the presence of Con A (concavaline-A) and in the absence of CEP and APC (white bars). Flow cytometry analysis have shown that the CD4+IL4+/CD4+IFNγ+ ratio decreased significantly in tolerized mice in groups B and E and in the control groups C and F, and increased significantly in non-tolerized (n-CEP) mice in groups A and D.

Evaluation of the effect of NK1.1 depletion in the absence of the antigen showed a similar effect to the one described in the presence of antigen (black bars). Lymphocytes harvested from tolerized mice in group B revealed significantly higher CD4+IL4+/CD4+IFNγ+ ratio compared with NK1.1-depleted mice in tolerized group E. In contrast, NK1.1 depletion induced an increase in the CD4+IL4+/CD4+IFNγ+ ratio in non-tolerized mice from groups A and D in the absence of the disease target antigen. Abbreviations: EXP. GR.=Experimental groups, rat.=ratio, CEP=colitis extracted protein, n-CEP.=non-tolerized.

FIG. 6: IL4 and IFNγ levels in the different experimental groups

Supernatant fluids were collected from both sets of triplicates and cytokine levels were measured for all mice from all tolerized and non-tolerized groups (different groups are indicated by A, B, C, D, E, F). IL4 and IFNγ levels were measured by a “sandwich” ELISA. Tolerized mice manifested a shift from Th1 to Th2 immune response cytokine secretion. These mice (group B) manifested an increase in IL4 levels and a decrease in IFNγ levels. In contrast, mice from non-tolerized groups (groups A, E and F) exhibited high IFNγ and low IL4 levels. Abbreviations: EXP. GR.=Experimental groups.

FIG. 7: Effect of NK1.1− depletion on IL12 levels

Supernatant fluids were collected from both sets of triplicates and cytokine levels were measured for all mice from all tolerized and non-tolerized groups (different groups are indicated by A, B, C, D, E, F). NK1.1 depletion led to an increase in IL12 levels in the CEP-fed groups (groups E and B, respectively) but had an opposite effect in the non-CEP fed groups (groups A and D). Abbreviations: EXP. GR.=Experimental groups.

FIG. 8A-8B: Effect of tolerization on histologic evaluation of bowel mucosa in experimental colitis.

FIG. 8A: shows paraffin sections from distal colonic tissue (last 10 cm) of non-tolerized mice.

FIG. 8B: shows paraffin sections from distal colonic tissue (last 10 cm) of tolerized mice.

Sections were stained with hematoxylin-eosin. Feeding of mouse-derived CEP led to marked alleviation of experimental colitis, manifested by marked reduction in inflammatory response and mucosal damage (group H, FIG. 8B). In contrast, severe colitis was observed in non-tolerized mice fed with BSA (group G, FIG. 8A).

FIG. 9: NK1.1+ lymphocytes increase the CD4+IL4+/CD4+IFNγ+ ratio in tolerized mice

Splenocytes and liver-associated-lymphocytes (2.5×10⁶ splenocytes and 0.5×10⁶ LAL) were harvested from mice in all groups and cultured for 72 hours in the presence of CEP and APC. The different experimental groups are indicated by G′, H′, I′, J′, K′ and L′. Flow cytometry analysis have shown that NK1.1-LAL depletion following oral tolerance induction decreased the CD4+IL4+/CD4+IFNγ+ ratio (group H′) in comparison with the non-NK1.1 depleted tolerized mice (group K′). Control NK1.1-depleted group (group L′) had a decrease in CD4+IL4+/CD4+IFNγ+ ratio compared with non-NK1.1-depleted group (group I′). NK1.1+ lymphocytes decreased the CD4+IL4+/CD4+IFNγ+ ratio in non-tolerized mice with experimental colitis. In contrast to tolerized groups, NK1.1-depletion had an opposite effect in non-tolerized mice with experimental colitis The CD4+IL4+/CD4+IFNγ+ ratio increased in NK1.1-depleted non tolerized group (group J′), in comparison with the non-NK1.1 depleted non-tolerzed group (groups G′). Abbreviations: EXP. GR.=Experimental groups, rat.=ratio.

FIG. 10: Expression of IL4 and IFNγ on isolated lymphocytes from different experimental groups

The figure shows representative results of flow cytometry analysis for determination of IL4 and IFNγ expression. Expression of IL4 and IFNγ on. isolated lymphocytes from tolerized NK1.1 non-depleted and depleted mice, and non-tolerized NK1.1 non-depleted and depleted mice. Data are displayed as dot plots after gating of 5×10⁴ small lymphocytes. Numbers below dot plots represent the percentages of stained cells. Representative results are shown. Experimental groups (EXP GR).

The different experimental groups are indicated by G, H, I and J.Abbreviations: EXP. GR.=Experimental groups, rat.=ratio.

FIG. 11: liver lymphocytes cytotoxicity by NK1.1

YAC-1 cells were used as target cells in these studies at an E:T ratio of from 100:1 t0 10:1. Recipients from non-tolerized non-NK1.1 depleted mice (group H′) showed almost no lysis compared to the other groups. Recipients from non-tolerized NK1.1− depleted mice in group G′ showed higher lysis then group H′, respectively. Recipients from NK1.1- depleted CEP fed mice from group I′ showed lower lysis then non NK1.1 depleted mice in group J′. Recipients from control groups had 23% vs. 22.47% cytotoxicity, for mice in group K′ compared with group L′ respectively. The different experimental groups are indicated by G′, H′, I′, J′, K′ and L′. .Abbreviations: EXP. GR.=Experimental groups.

FIG. 12: Cytokine levels in different experimental groups

Supernatant fluids were collected from both sets of triplicates and cytokine levels were measured for all mice from all tolerized and non-tolerized groups. IL4, IL10, and IFNγ levels were measured by a “sandwich” ELISA. Tolerized mice manifested a shift from Th1 to Th2 immune response cytokine secretion. These mice (group H) manifested an increase in IL4, IL10 levels and a decrease in IFNγ levels. In contrast, mice from non-tolerized groups (groups G, J, K) and control group I, exhibited high IFNγ and low IL10 levels. Lymphocytes harvested from tolerized mice in group H revealed significantly higher IL4, IL10, and lower IFNγ levels compared with NK1.1-depleted mice in tolerized group K. In contrast, NK1.1 depletion induced an increase in IFNγ and a decrease in IL4, IL10 levels in non-tolerized mice from groups G and J in the absence of antigen. The different experimental groups are indicated by G, H, J and K. Abbreviations: EXP. GR.=Experimental groups. IL4 and IL10 are indicated by black bars and IFNγ by white bars.

### DETAILED DESCRIPTION OF THE INVENTION

NK1.1 T cells may be involved in keeping a balance between anti-inflammatory and pro-inflammatory lymphocytes via cytokines secretion, and/or killing, and may be involved in the determination of T helper cell differentiation [Arase, H., et al., Eur. J. Immunol. 23: 307-310 (1993); Yoshimoto, T., et al., J. Exp. Med. 179:1285-1295 (1994), MacDonald, H. R., et al., J. Exp. Med. 182:633-638 (1995), Seder, R. A. et al., Annu. Rev. Immuno. 12:635-673 (1994), Yoshimoto, T., et al., Science 270:1845-1847 (1995)]. Multiple signaling pathways were identified for NK1.1 T cells activation. It is assumed that NK1.1+ T cells are not stably polarized, and upon different triggers TCR engagement triggers both Th1 and Th2 cytokine secretion from these cells [Bendelac, A., et al., Annu. Rev. Immunol. 15:535-562 (1997); Arase, H., et al., J. Immunol. 151:546 (1993); Kawamura, T., et al., J. Immunol. 160:16-19 (1998), Chen, H., et al., J. Immonol., 159:2240-2249 (1997); Arase, H., et al., Eur. J. Immunol. 23: 307-310 (1998);Yoshimoto, T., J. Exp. Med. 179: 1285-1295 (1994); MacDonald, H. R., J. ibid., (1995)]. NK1.1R or IL12R engagement may selectively promote the Th1 secretion paradigm [Bendelac, et al. (1997) ibid.; Arase, H., et al., J. Exp. Med. 183:2391-2396 (1996); Hayakawa, T., et al., J. Exp. Med. 176:269-274 (1992)].

As described above, NK1.1+ T lymphocytes play a complicated role in immunoregulation. The results described in the present invention show that NK1.1 T lymphocytes have a dual role in regulating immune-mediated experimental colitis. On the one hand, depletion of NK1.1 T lymphocyte following oral tolerance induction prevented the adoptive transfer of tolerance, while significantly decreasing the quantitative ratio between IL4 to IFNγ secreted by CD4⁺ cells. On the other hand, depletion of NK1.1 T lymphocyte in non-tolerized mice, alleviated colitis and significantly increased the quantitative ratio between IL4 secreted by CD4⁺ to IFNγ secreted by CD4⁺.

In a first aspect, the invention thus relates to a method for the treatment of immune-related disorders in a mammalian subject in need of such treatment. The method of the invention comprises the step of manipulating the NK T cell population in a subject by suitable means. The manipulation of the NK T cell population results in modulation of the Th1/Th2 cell balance and shifts it toward the production of anti-inflammatory cytokine producing cells. It should be emphasized that any immune-modulation can down or up regulate the immune response. This modulation is further mediated by different components of the subject's immune system. Such components are, for example, cellular immune reaction elements, humoral immune reaction elements and cytokines.

In a preferred embodiment, manipulating the NK T cell population is by depletion of this cell population. Depletion of the NK T cell population may be performed, for example, by administering to the subject a therapeutically effective amount of a composition comprising as the effective ingredient an antibody that specifically recognizes the NK T cells. This specific method encompasses the use of polyclonal as well as, preferably monoclonal antibodies.

The generation of polyclonal antibodies against proteins is described in Chapter 2 of Current Protocols in Immunology, Wiley and Sons Inc. Monoclonal antibodies may be prepared from B cells taken from the spleen or lymph nodes of immunized animals, in particular rats or mice, by fusion with immortalized B cells under conditions which favor the growth of hybrid cells. For fusion of murine B cells, the cell line Ag-8 is preferred. The technique of generating monoclonal antibodies is described in many articles and textbooks, such as the above-noted Chapter 2 of Current Protocols in Immunology. Spleen or lymph node cells of these animals may be used in the same way as spleen or lymph node cells of protein-immunized animals, for the generation of monoclonal antibodies as described in Chapter 2 therein. The techniques used in generating monoclonal antibodies are further described by Kohler and Milstein, Nature 256:495-497, (1975), and in U.S. Pat. No. 4,376,110.

The term “antibody” is meant to include both intact molecules as well as fragments thereof, such as, for example, Fab and F(ab′)₂, which are capable of binding antigen. Fab and F(ab′)₂ fragments lack the Fc fragment of intact antibody, clear more rapidly from the circulation, and may have less non-specific tissue binding than an intact antibody [Wahl et al., J. Nucl. Med. 24: 316-325, (1983)]. It will be appreciated that Fab and F(ab′)₂ and other fragments of the antibodies useful in the present invention may be used for the selective depletion of the NK T cells, according to the methods disclosed herein for intact antibody molecules. Such fragments are typically produced by proteolytic cleavage, using enzymes such as papain (to produce Fab fragments) or pepsin (to produce F(ab′)₂ fragments).

An antibody is said to be “capable of specifically recognizing” a certain cell if it is capable of specifically reacting with an antigen which is in this particular example an extracellular marker molecule expressed by said cell, to thereby bind the molecule to the antibody.

An “antigen” is a molecule or a portion of a molecule capable of being bound by an antibody, which is additionally capable of inducing an animal to produce antibody capable of binding to an epitope of that antigen. An antigen may have one or more than one epitope. The term “epitope” is meant to refer to that portion of any molecule capable of being bound by an antibody that can also be recognized by that antibody. Epitopes or “antigenic determinants” usually consist of chemically active surface groupings of molecules such as amino acids or sugar side chains, and have specific three-dimensional structural characteristics as well as specific charge characteristics.

As an alternative, depletion of the NK T cell population may be performed by an ex vivo pheresis, using beads coated with an antibody that specifically recognizes the NK T cells. In a pheresis procedure the whole blood is drawn from the treated subject, and is immediately separated into plasma, red cells and white cells. The NK T cells are depleted from the white cells population, by using a specific antibody to the NK T cell markers, while other blood components are being simultaneously transferred back to the treated subject.

NK1.1 molecules on NK1.1⁺ T cells serve as receptors leading to IFNγ and not to IL4 production [Arase, H., et al., J. Exp. Med. 183:2391-2396 (1996); Seder, R. A., et al., Proc. Natl. Acad. Sci. USA 90:10188-92 (1993)]. Upon stimulation with glycosylposphatidylinositol-anchored protein or LPS ligand, NK1.1 T cells become IFNα producing cells, inhibit Th2 cell differentiation and suppress IgE response [Cui, J., et al., J. Exp. Med. 190(N-6): 783-792 (1999)]. Exogenous IL2 increases IFNγ production upon NK1.1R-P1 cross-linking [Arase, H., et al., (1996) ibid.]. NK1.1⁺ T cells are involved in CD4⁺ T cells differentiation via secretion of large amount of IL4 promptly upon in vivo stimulation with anti-CD3 [Yoshimoto, T., et al., Science 270:1845-7 (1995)]. CD1-restricted NK1.1 T cells population is essential for anti-CD3-induced early IL4 burst [Seder, R. A., Ann. Rev. Imm. 12:635-673 (1994)]. Bacterial LPS has been shown to activate NK1.1⁺ cells via IL-12 production from Kupffer cells and subsequently induces IFNγ production [Ma, X., et al., J. Exp. Med. 183:147-157(1996)]. Cell to cell contact between dendritic cells and NK and/or T lymphocytes resulted in a substantial increase in both cell cytolytic activity and IFNα production [De-Moraes, L., et al., Eur. J. Immunol. 28:1507-1515 (1998)]. IL18 and leukocyte function-associated antigen-1 may play a role in the accumulation of NK1.1⁺ T cells in the liver and in their cytotoxic activity [Sakamoto, Y. et al., J. Immunol., 103(5 pt 2):445-51 (1999)]. NK1.1+ T cells have been suggested as playing a role in antigen presentation, which may be another pathway by which they influence T cell response [Seki. S., et al., J. Immunol. V 147:1214-1221 (1991)]. This subtype of cells was previously shown to have a high level of autologous killing [Crispe, N., et al., Immun. Today 11:236-245 (1996); Kawamura, T., et al., J. Immunol. 160:16-19 (1998); Dohert, D. G., et al., J. Hepatology 28:59A. (1998)]. Fas expression by LAL resulted in death of activated Fas expressing T cells [Dohert, D. G., et al., (1998) ibid.; Jonsson, J. R., et al., Hepatology 26:269A(1997); Doherty, D. G., et al., Hepatology 26: 445A (1997)]. Thus it is possible that in a tolerized environment NK1.1 T cells may be involved in killing sensitized pro-inflammatory cells in addition to their IL4-mediated anti-inflammatory cytokines secretion, whereas in a non-tolerized environment they may be involved in killing anti-inflammatory cells in addition to their IFNγ secretion. Both IL4 and IL12 increase the cytotoxic potential of NK1.1 T cells [Hashimoto, W., et al., J. Immonol. 154: 4333-4340 (1995); Ballas, Z. K., et al., J. Immonol. 150:17-30 (1993)]. During inflammation there is an IL12/IFNγ loop which plays a role in balancing the immune response [Ma, et al., (1996) ibid.]. IL12 augments IFNγ secretion, as well as the cytolytic activity and proliferation of NK1.1+ T cells [Cui, et al., (1999) ibid.; Bendelac et al., (1997) ibid.; Arase, et al., (1996) ibid., De-Moraes, et al., (1998) ibid.; Neurath, M. F., et al., J. Exp. Med., 182:1281-1290 (1995)].

Therefore, as an alternative most preferred embodiment, the invention relates to a method for treatment of immune-related disorders in a mammalian subject. This method involves manipulation of NK T cell population by ex vivo education of said NK T cells, such that the educated NK T cells have the capability of modulating the Th1/Th2 balance and shifting it toward the production of anti-inflammatory cytokine producing cells and administration of the educated cells into said subject. This modulation results in an increase in the quantitative ratio between any one of IL4 and IL10 to IFNγ (may also be referred throughout the specification as CD4⁺IL4, IL10/CD4⁺IFNγ ratio). In immune-mediated disorders the ratio decreases according to severity of the disease and it may increase during recovery. Therefore, it is to be appreciated that the change in the quantitative ratio between any one of IL4 and IL10 to IFNγ should be related to the pre-treatment level.

The term “CD4⁺IL4” is meant the IL4 produced by CD4⁺ cells,“CD4⁺IL10” is meant the IL10 produced by CD4⁺ cells and “CD4^{+IFNγ” is meant the IFNγ produced by CD}4⁺cells. The term “CD4⁺IL4 IL10/CD4⁺IFNγ ratio” used in the present invention, is meant the quantitative ratio between any one of IL4 and IL10 preferably produced by CD4⁺ cells, and between the IFNγ preferably produced by CD4⁺ cells. Quantitative measurements for defining the quantity of each of these cytokines were performed as described in the Examples (experimental procedures).

A specifically preferred embodiment relates to method for the treatment of immune-related disorders in a mammalian subject. The method of treatment comprises the steps of:

a. obtaining NK T cells from said subject;

b. ex-vivo educating the NK T cells obtained in step (a) such that the resulting educated NK T cells have the capability of modulating the Th1/Th2 cell balance toward anti-inflammatory cytokine producing cells; and

c. re-introducing to said subject the educated NK T cells that were obtained in step (b). Modulation of the Th1/Th2 balance toward anti-inflammatory cytokine producing cells, results in increase in the quantitative ratio between any one of IL4 and IL10 to IFNγ.

NK T cells can be obtained from bone marrow, liver, spleen, or uterus, but can also be obtained from the peripheral blood, by cytopheresis methods as described above.

More specifically, ex-vivo education of the NK T cells may be performed by culturing these cells in the presence of any one of:

a. at least one of antigens associated with, bystander epitopes to the immune-related disorder to be treated or any combinations thereof;

b. at least one of liver-associated cells of tolerized or non-tolerized patients suffering from the same immune disorder or from the subject to be treated or any combination thereof;

C. at least one of cytokines, adhesion molecules or any combination thereof, and

d. a combination of any of (a), (b) and (c) above.

It is to be appreciated that the NK T cells may be educated in vivo as well, via any of the methods described above, they can be modulated prior to or at any point of time following exposure to the allogeneic epitopes or antigens.

In one particular embodiment, the ex vivo education of the NK T cells may be performed by culturing these cells in the presence of antigens associated with the immune-related disorder to be treated. These antigens in may be allogeneic antigens taken from donor patients suffering from said immune-related disorder, xenogeneic antigens, autologous antigens and recombinantly prepared antigens or any combinations thereof.

These antigens can be native or non-native with regards to the subject. They can be natural or synthetic, modified or unmodified, whole or fragments thereof. Fragments can be derived from synthesis as fragments or by digestion or other means of modification to create fragments from larger entities. Such antigen or antigens comprise but are not limited to proteins, glycoproteins, enzymes, antibodies, histocompatibility determinants, ligands, receptors, hormones, cytokines, cell membranes, cell components, viruses, viral components, viral vectors, non-viral vectors, whole cells, tissues or organs. The antigen can consist of single molecules or mixtures of diverse individual molecules. The antigen can present itself within the context of viral surface, cellular surface, membrane, matrix, or complex or conjugated with a receptor, ligand, antibody or any other binding partner. Such antigen or antigen can be introduced to the subject alone or with agent or agents that could further contribute to uptake, stability, reactivity or targeting.

Polymerization and degradation, fractionation and chemical modification are all capable of altering the properties of a particular antigen in terms of potential immune responses. These small segments, fragments or epitopes can either be isolated or synthesized.

As a non-limiting example, such antigen may be combination of different antigens derived from body extracts, such as the CEP used for ex vivo education in Example 7.

The method of the present invention, further encompasses recombinantly prepared antigens. Preparation of recombinant antigens involves the use of general molecular biology techniques that are well known in the art. Such techniques include for example, cloning of a desired antigen to a suitable expression vector.

“Vectors”, as used herein, encompass plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or an eukaryotic promoter expression control system. This typically include a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector-containing cells. Plasmids are the most commonly used form of vector but other forms of vectors which serves an equivalent function and which are, or become, known in the art are suitable for use herein. See, e.g., Pouwels et al., Cloning Vectors: a Laboratory Manual (1985 and supplements), Elsevier, N. Y.; and Rodriquez, et al. (eds.) Vectors: a Survey of Molecular Cloning Vectors and their Uses, Buttersworth, Boston, Mass. (1988), which are incorporated herein by reference.

It has been recently proposed that the liver is a major site of T cell destruction and that in the liver of autoimmune mice lpr/lpr, there is a failure of this process with leakage of T cells from the liver to peripheral lymphoid tissues [Crispe, N., et al., Immunol. Review, 174:47-62 (2000)]. The liver was shown to play a role in T cell differentiation. CD3CD4⁺/CD8⁺TCRβ cells and CD3-4-TCRβ⁺ cells can be generated from CD4-8-TCRβ athymic nude bone marrow cells by culture with liver parenchymal cells [Mabuchi, A., et al., J. Leukocyte Biology, 63:575-583 (1998)]. Therefore, in another particular embodiment, the ex vivo education of the NK T cells may be performed by culturing these cells in the presence of liver-associated cells. These cells may be for example Kupffer cells, Stellate cells, liver endothelial cells liver associated stem cells or any other liver-related lymphocytes.

Co-culturing of the NK T cells in the presence of peripheral lymphocytes from tolerized or non-tolerized patients suffering from the same immune-related disorder or from the treated subject, is also contemplated in the present invention. In order to obtain lymphocytes from a subject, particularly human subject, blood is drawn from the patient by cytopheresis, a procedure by which a large number of white cells are obtained, while other blood components are being simultaneously transferred back to the subject.

As described in Example 7, ex vivo education of NK T cells may be preformed by co-culturing of NK T cells with CD4 or CD8 cells. These cells are preferably obtained from a tolerized subject (mice received CEP as oral tolarization).

In another particular embodiment, the ex-vivo education of the NK T cells may be performed by culturing the cells in the presence of cytokines such as IL4, IL10, TGFβ, IFNγ, IL12 and IL15, or in the presence of adhesion molecules such as Integrins, Selectin and ICAM.

While IL12 exerts an effect of IFNγ induction by NK1.1 T cells, IFNγ may in turn contribute to its regulation [Ma et al., (1996) ibid.]. Cytolytic activity of thymocytes from mice undergoing acute GVHD, decreased significantly following NK1.1⁺ cell depletion [Neurath et al., (1995) ibid.]. The increase in NK1.1⁺ T cells in the thymus of mice suffering from acute GVHD, was preceded by a transient increase of IL12 production in the thymus [Neurath et al., (1995) ibid.]. IL12 was reported to induce an increase of NK1.1+ T cells in the thymus of mice suffering from acute GVHD [Onoe, Y., et al., Immunology 95:248-256. (1998)]. It was recently shown that anti-IL12 antibodies enhance oral tolerance in transgenic animals and was associated with increased TGFβ, secretion [Marth, T., et al., J. Immunol. 157:2348-2357 (1996)]. Both IL12 and TNFα were shown to have an important role in the immunepathogenesis of experimental colitis [Bragger, M. S. H., et al., Gut 34:1705 (1998); Parronchi, P., et al., Am. J. Pathol. 150:823 (1997)]. IL12 production by monocytes/macrophages was essential in maintaining TNBS induced colitis and was required for the Th1-mediated inflammatory response [Kuhn, R., et al., Cell 75:263-274, (1993); Sellon, R. K., et al., Immun. 66:5224-5231 (1998); Neurath et al (1995) ibid.; Marth, T., et al., J. Immunol. 157:2348-2357 (1996)].

Antibodies to IL12 abrogated chronic TNBS induced colitis [Neurath et al., (1995) ibid.]. Therefore, IL12 may have a dominant role in disease pathogenesis via NK1.1⁺ T cell activation. It is possible that activation of this subset of lymphocytes induces IFNγ secretion, followed by a Th1 immune shift in non-tolerized mice [Arase, et al., (1996) ibid.; Bleicher, P. A., et al., Science 250:679-682 (1990); Kitamura, H., et al., J. Exp. Med. 189:1121-1127 (1999)].

NK1.1⁺ T cells may be potent IFNγ producers in the presence of IL12 in the experimental colitis [Cui et al., (1999) ibid.; Bendelac et al., (1997) ibid.; Arase et al., (1996) ibid.; De-Moraes et al., (1998) ibid.]. The results of the present invention suggest that IFNγ was secreted by NK1.1 T cells in the inflammatory state via NK1.1R, independent of the IL12 pathway. This may have been followed by IFNγ triggered-IL12 production, with IL12 induced-IFNγ secretion via the IL12R. In contrast, in the anti-inflammatory tolerized state, NK1.1 T cell are activated with increased IL4 secretion. Indeed, adoptive transfer of lymphocytes from non-tolerized NK1.1-depleted mice upregulated the anti-inflammatory Th2 cytokines. It is possible that different stimuli determine the type of cytokine response.

Thus, chemokines or other mediators may determine NK1.1+ T cell function and the way in which they influence the Th1/Th2 paradigm in different immunological environments.

In a specifically preferred embodiment, the NK T cell that has been ex vivo educated as described above may be re-introduced to the treated subject. This can be carried out by a process that has been termed adoptive transfer. The particular educated NK T cells used for the transfer may preferably originate from the subject (autologous transfer). A syngeneic or non-syngeneic donor (non-autologous transfer) is not excluded. The storage, growth or expansion of the transferred cells may have taken place in vivo, ex vivo or in vitro.

Methods for in vitro storage, growth or expansion of cells prior to transfer are well known to practitioners of the art. When the educated NK T cells intended for use in a transfer are derived from a donor, these cells may also undergo storage, growth or expansion in vivo or in vitro as described above.

Cell therapy may be by injection, e.g., intravenously, or by any of the means described herein above. Neither the time nor the mode of administration is a limitation on the present invention. Cell therapy regimens may be readily adjusted taking into account such factors as the possible cytotoxicity of the educated cells, the stage of the disease and the condition of the patient, among other considerations known to those of skill in the art.

The method of the invention may optionally further comprises the step of eliciting in the treated subject up or down regulation of the immune response to the immune-related disorder. A down regulation response may be achieved by administering to said subject components, cells, tissues or organs derived from any one of allogeneic donors suffering from said immune-related disorder, xenogenic sources and autologous sources, and immunologically equivalents, or any combinations thereof.

The present invention provides for the administration of non-native active compounds without the risk of an immune response that could diminish the effectiveness of such treatment, whether such treatment is transient or whether such treatment is made repeatedly over a prolonged period. The present invention thus provides for the effective biological function of these non-native active compounds without interference by the body's immune response. This can be achieved by the use of the immune modulation as provided in this invention wherein it can be used as general immune suppression for transient or short term treatment and/or by tolerization, provided by modulation of the immune response, for prolonged treatment. In some cases a combination of two or more such immune-modulation regimens can be advantageous. Such treatments can be applied prior to and/or during the course of administration of non-native active compounds.

In a specifically preferred embodiment, the said components, cells, tissues or organs may by administered in a single dose, or alternatively in multiple doses. These components, cells, tissues or organs may be administered by a single route of administration or alternatively, by at least two different routes of administration.

The components may be administered directly to the subject to be treated or, depending on the size of the compound, it may be desirable to conjugate them to a carrier prior to their administration. Therapeutic formulations may be administered in any conventional dosage formulation. Formulations typically comprise at least one active ingredient, as defined above, together with one or more acceptable carriers thereof

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

More specifically, the said components, cells, tissues or organs may be administered by a route selected from oral, intravenous, parenteral, transdermal, subcutaneous, intravaginal, intranasal, mucosal, sublingual, topical and rectal administration and any combinations thereof. Preferably, these components, cells, tissues or organs are administered orally as an oral tolarization.

Another preferred embodiment of the method of the invention relates to the treatment of an inflammatory bowel disease (IBD), more particularly Crohn's disease. The treatment of Crohn's disease in a mammalian, particularly human subject, comprises the steps of:

a. obtaining NK T cells from said subject;

b. ex vivo educating the NK T cells obtained in step (a) such that the resulting educated NK T cells have the capability of modulating the Th1/Th2 cell balance toward anti-inflammatory cytokine producing cells; and

c. re-introducing to said subject the educated NK T cells that were obtained in step (b). Modulation of the Th1/Th2 balance toward the production of anti-inflammatory cytokine producing cells results in increase in the quantitative ratio between any one of IL4 and IL10 to IFNγ.

Although the method of the invention is particularly intended for the treatment of immune-related disorders in humans, other mammals are included. By way of non-limiting examples, mammalian subjects include monkeys, equines, cattle, canines, felines, mice, rats and pigs.

For treating a human patient, the method of the invention may utilize for ex vivo education a specific subtype of NK T cells, which is the NK T cells that express the CD56 marker. For mice, the method of the present invention may utilize for ex vivo education the specific subtype of NK 1.1⁺ T cells. The Examples of the present invention disclose experiments using the NK 1.1⁺ cells of a mouse model. It is to be appreciated that these results are also applicable to the NK T cells that express the CD56 marker, in humans. The ex vivo educating of the CD56 marker expressing NK T cells according to the invention is by culturing these cells in the presence of any one of:

a. at least one of antigens associated with Crohn's disease; these antigens may be for example allogeneic antigens from donors suffering of Crohn's disease, xenogenic antigens, autologous antigens from said patient itself and recombinantly prepared or any combinations thereof,

b. at least one of liver-associated cells from tolerized or non-tolerized patients suffering from Crohn's disease or from the treated patient; these cells may be for example Kupffer cells, Stellate cells, liver endothelial cells liver associated stem cells or any other liver-related lymphocytes or any combinations thereof;

c. at least one of cytokines such as IL4, IL10, TGFβ IFNγ IL12 and IL15, or adhesion molecules such as Integrins, Selectin and ICAM or any combinations thereof; and

d. a combination of any of (a), (b) and (c) above.

The educated NK T cell according to the method of the invention is re-introduced by adoptive transfer to the treated subject.

The method of the invention may optionally further comprises the step of eliciting in the subject up or down regulation of the immune response to inflamed intestine. The elicitation of down regulation response may be induced by administering to the subject components that may be proteins extracted from inflamed intestines of a subject suffering from Crohn's disease, or from the intestines of the treated subject.

The components, may be cells, tissues or organs or parts thereof and they may be administered in a single dose, or alternatively in multiple doses. These components may be administered by a single route of administration or alternatively, by at least two different routes of administration. More specifically, said components may be administered by a route selected from oral, intravenous, parenteral, transdermal, subcutaneous, intravaginal, intranasal, mucosal, sublingual, topical and rectal administration and any combinations thereof. Preferably, the components are administered orally as oral tolarization (oral introduction of CEP) as described in the Examples.

In another specifically preferred embodiment, the method of the invention is intended for the treatment of a malignancy. In cancerous situations, modulation of the NK T cells may be in the direction of inducing a pro-inflammatory response or in augmenting the anti-tumor associated antigens immunity. As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods and compositions of the present invention may be used in the treatment of non-solid and solid tumors.

Malignancy, as contemplated in the present invention may be selected from the group consisting of melanomas, carcinomas, lymphomas and sarcomas. Malignancies that may find utility in the present invention can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including lung, liver, breast, colon, prostate GI tract, pancreas and Karposi).

For treating a mammalian subject suffering of cancer, the educated NK T cell used by the method of the invention can be administered in a variety of ways. By way of a non-limiting example, the educated cells may be delivered intravenously, or into a body cavity adjacent to the location of a solid tumor, such as the intraperitoneal cavity, or injected directly into or adjacent to a solid tumor.

Still further, the present invention provides for a method for education of NK T cells. This education may be performed by culturing these cells in the presence of any one of:

a. at least one of antigens associated with, bystander epitopes to the immune-related disorder to be treated or any combinations thereof,

b. at least one of liver-associated cells of tolerized or non-tolerized patients suffering from the same immune disorder or from the subject to be treated or any combination thereof,

c. at least one of cytokines, adhesion molecules or any combination thereof, and

d. a combination of any of (a), (b) and (c) above.

The methods of the invention may be combined with other therapies useful in the treatment of cancer. It is also anticipated that this treatment may be given to a mammalian subject that is already immuno-suppressed due to disease. The evaluation of the immune status of the human or veterinary patient may be readily determined by one of skill in the art.

As a second aspect the present invention relates to therapeutic composition for the treatment of immune-related disorder in a mammalian subject. The composition of the invention comprises as an effective ingredient ex vivo educated autologous NK T cells capable of modulating the Th1/Th2 balance toward anti-inflammatory cytokine producing cells. These educated autologous NK T cells mediate increase in the quantitative ratio between any one of IL4 and IL10 to IFNγ.

The compositions of the invention may further contain a pharmaceutically acceptable carrier, additive, diluent or excipient. Suitable carriers include, e.g., saline phosphate buffered saline, and saline with 5% HSA or PPF. Other suitable carriers are well known to those of skill in the art and are not a limitation on the present invention. Similarly, one of skill in the art may readily select other desired components for inclusion in a pharmaceutical composition of the invention, and such components are not a limitation of the present invention.

In a preferred embodiment, the educated autologous NK T cell of the therapeutic composition of the invention are ex vivo cultured in the presence of any one of:

a. at least one of antigens associated with the immune-related disorder to be treated that may be any one of allogeneic antigens from donors suffering from the same immune-related disorder, xenogenic antigens, autologous antigens from the treated patient and recombinantly prepared or any combinations thereof;

b. at least one of liver-associated cells of tolerized or non-tolerized patients suffering from said immune-related disorder or from the treated subject, which may be Kupffer cells, Stellate cells, liver endothelial cells, any other liver-related lymphocytes and any combination thereof,

c. at least one cytokines such as IL4, IL10, TGFβ IFNγ IL12 and IL15, or adhesion molecules such as Integrins, Selectin and ICAM; and

d. a combination of any of (a), (b) and (c) above.

In one preferred embodiment the therapeutic composition of the invention is intended for the treatment of intestinal inflammatory disease in a mammalian subject, and more specifically for the treatment of Crohn's disease. This composition comprises as an effective ingredient educated autologous NK T cells, which have been rendered capable of modulating the Th1/Th2 balance toward the production of anti-inflammatory cytokine producing cells.

The educated autologous NK T cell contained in the therapeutic composition of the invention is capable of modulating the Th1/Th2 balance and shift it toward the production of anti-inflammatory cytokine producing cells. The result of this balance shift is an increase in the CD4+IL4+/CD4+IFNγ ratio (the quantitative ratio between any one of IL4 and IL10 to IFNγ). These modulation processes are further mediated by different components of the subject's immune system, such as cellular immune reaction elements, humoral immune reaction elements and cytokines.

The education of the autologous NK T cell contained in the compositions is preferably performed as described above.

In another preferred embodiment the therapeutic composition of the invention is intended for the treatment of a malignancy such as melanoma, carcinoma, lymphoma and/or sarcoma. In cancerous situations, modulation of the NK T cells contained in the compositions of the invention may be in the direction of inducing a pro-inflammatory response or in augmentation of the anti-tumor associated antigens immunity.

In yet another preferred embodiment the invention relates to a therapeutic composition for the treatment of immune-related disorders. This composition comprises as an effective ingredient an antibody that specifically recognizes the NK T cells. The compositions of the invention may further contain a pharmaceutically acceptable carrier. Suitable carriers include, e.g., saline phosphate buffered saline, and saline with 5% HSA or PPF. Other suitable carriers are well known to those of skill in the art and are not a limitation on the present invention. Similarly, one of skill in the art may readily select other desired components for inclusion in a pharmaceutical composition of the invention, and such components are not a limitation of the present invention.

In one embodiment this therapeutic composition of the invention may be used for the treatment of an intestinal inflammatory disease, such as Crohn's disease. For the treatment of intestinal inflammatory diseases, and particularly Crohn's disease, oral pharmaceutical compositions may advantageous. Oral administration will permit amelioration of the patient's condition, without the need for systemic immunosuppression or invasive procedures.

In another embodiment the therapeutic composition of the invention may be used for the treatment of a malignancy selected from the group consisting of melanomas, carcinomas, lymphomas and sarcomas.

Composition dosages may be in any amount that sufficient to modulate the Th1/Th2 balance. It is understood by the skilled artisan that the preferred dosage would be individualized to the patient following good laboratory practices and standard medical practices.

As used herein, “an amount sufficient to modulate the Th1/Th2 balance” means an amount necessary to achieve a selected result. For example, an effective amount of the composition of the invention will modulate the Th1/Th2 balance toward anti-inflammatory cytokine producing cells.

The compositions and methods of the present invention may further provide for the treatment of autoimmune diseases such as insulin-dependent diabetes mellitus (IDDM).

The compositions of the invention can be administered in a variety of ways. By way of non-limiting example, the composition may be delivered intravenously.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The pharmaceutical compositions of the invention generally comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

As a third aspect, the present invention relates to the use of an educated autologous NK T cell, in the manufacture of therapeutic pharmaceutical compositions for modulating the Th1/Th2 cell balance toward the preferred production of anti-inflammatory cytokine producing cells, in a mammalian subject suffering of a immune-related disorder. Preferred use is the manufacture of compositions for the treatment of intestinal inflammatory disease in a mammalian subject, more specifically, Crohn's disease in human subjects. Alternatively, the educated autologous NK T cells may be used in the preparation of therapeutic pharmaceutical compositions for the treatment of a malignancy, such as melanoma, carcinoma, lymphoma and sarcoma. In cancerous situation, modulation of the NK T cells of the invention may be in the direction of inducing a pro-inflammatory response or in augmentation of the anti-tumor associated antigens immunity, in a favorable direction.

The present invention further provides for an ex vivo educated autologous NK T cell. The educated NK T cell has been ex vivo cultured in the presence of any one of:

a. at least one of antigens associated with said immune-related disorder or any combinations thereof;

b. at least one of liver-associated cells of tolerized or non-tolerized patients suffering from said immune-related disorder or of said subject or any combinations thereof;

c. at least one of cytokines, or adhesion molecules; and

d. a combination of any of (a), (b) and (c) above.

Still further, the invention provides for an ex vivo educated autologous NK T of the invention for use in the treatment of immune-related disorders in a mammalian subject in need of such treatment.

In another embodiment of the present aspect, the invention relates to the use of an ex vivo educated autologous NK T cell in the treatment of immune-related disorders in a mammalian subject in need of such treatment.

In yet another preferred embodiment the present invention relates to the use of an antibody that specifically recognizes the NK T cells, in the manufacture of a therapeutic pharmaceutical composition for manipulation of the NK T cells population in a mammalian subject suffering of a immune-related disorder, specifically depletion of said NK T cell population in said subject. It is to be appreciated that the depletion of the NK T cells population results in modulating the Th1/Th2 balance toward the preferred production of anti-inflammatory cytokine producing cells. The antibodies may be particularly used for the preparation of a therapeutic pharmaceutical composition for the treatment of immune-related disorder in a mammalian subject, specifically intestinal inflammatory disease, such as Crohn's disease in a human subject.

In another specific embodiment the immune-related disorder may be a malignancies such as melanomas, carcinomas, lymphomas and sarcomas.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

I.

Materials and Methods

Animals

Normal inbred 2 to 4 month old C57BL male mice were obtained from Harlan and maintained in the Animal Core of the Hadassah-Hebrew University Medical School. Mice were maintained on standard laboratory chow and kept in 12-hour light/dark cycles.

Induction of Colitis

TNBS-colitis was induced by rectal instillation of TNBS, 1 mg/mouse, dissolved in 100 ml of 50% ethanol as described. [Collins, C., et al., Eur. J. Immunol. 26:3114-3118 (1996)].

Preparation and Administration of the Oral Antigen

Colons were removed from TNBS-induced-colitis mice, cut into small strips, and mechanically homogenized. After filtration through a 40 mm nylon cell strainer, intact cells were spun down and removed. Proteins were quantified by using a protein assay kit (Biorad, Munich, Germany). Colitis extracted proteins (CEP) were introduced into the experimental groups described below by using a feeding atraumatic-needle every other day for 11 days (a total of 5 doses).

NK1.1 Cells Depletion

Depletion of NK1.1+ cells was performed by using mouse anti-mouse NK1.1 monoclonal antibody (Serotec, Oxford, UK) as previously described [Kawamura, T., et al., J. Immunol. 160:16-19 (1998)]. Mice were injected with 50 μg/day IP 36 hours before splenocyte harvesting from donor mice.

Adoptive Transfer of Lymphocytes

Donor mice from all groups were sacrificed 14 days after induction of colitis and single suspensions of lymphocytes derived from spleens were prepared as described [Weiner, H., et al., Annu. Rev. Immunol. 12:809-837 (1994)]. Cells were re-suspended in PBS before transplantation. Splenic lymphocytes from all groups were transplanted into naive recipient mice, followed 24 hours later by rectal challenge with TNBS.

Evaluation of the Effect of Tolerance Induction on Experimental Colitis

The effect of tolerance induction was evaluated by monitoring the following parameters for colitis:

Clinical assessment of colitis:

Diarrhea was followed daily throughout the study.

Macroscopic Score of Colitis

Colitis assessment was performed 14 days following colitis induction using standard parameters [Madsen, K. L., et al., Gastroenterology 113:151-159 (1997); Trop, S., et al., Hepatology 27:746-755 (1999)].

Four macroscopic parameters were determined, namely: degree of colonic ulcerations; intestinal and peritoneal adhesions; wall thickness; and degree of mucosal edema. Each parameter was graded on a scale from 0 (completely normal) to 4 (most severe) by two experienced blinded examiners.

Grading of Histological Lesions

For histological evaluation of inflammation, distal colonic tissue (last 10 cm) was removed and fixed in 10% formaldehyde. Five paraffin sections from each mouse were then stained with hematoxylin-eosin by using standard techniques. The degree of inflammation on microscopic cross sections of the colon was graded semiquantitatively from 0 to 4 [Madsen et al., (1997) ibid.; Trop et al., Hepatology 27:746-755 (1999)]. Grade 0: normal with no signs of inflammation; Grade 1: very low level of leukocyte infiltration; Grade 2: low level of leukocyte infiltration; and Grade 3: high level of infiltration with high vascular density, and bowel wall thickening; Grade 4: transmural infiltrates with loss of goblet cells, high vascular density, wall thickening, and disruption of normal bowel architecture. The grading was performed by two experienced blinded examiners.

Evaluation of the Role of NK1.1 Lymphocyte on Tolerance Induction in the Experimental Colitis Model

Liver and Spleen Lymphocytes Isolation

Splenocytes were isolated and red blood cells removed as previously described [Vicari, A. P., et al., Immunology Today 17(2):71 (1996)]. Intrahepatic lymphocytes were isolated from all groups of mice at the end of the study, as previously described, with some modifications [Vicari et al., (1996) ibid.; Bleicher, P. A., et al., Science 250:679-682 (1990)]. The inferior vena cava was cut above the diaphragm and the liver was flushed with 5 ml of cold PBS until it became pale. The connective tissue and the gal lbladder were removed, and livers were place in a 10-ml dish in cold sterile PBS. Livers and spleens were crushed through a stainless mesh (size 60, Sigma Chemical Co., St. Louis Mo.). Cell suspension was placed in a 50 ml tube for 3 minutes and washed twice in cold PBS (1,250×rpm for 10 minutes), and debris was removed. Cells were re-suspended in PBS, cell suspension was placed through a nylon mesh presoaked in PBS, and unbound cells were collected. Cells were washed twice in 45 ml PBS (1,250xrpm in room temperature). For liver and spleen lymphocyte isolation 20 ml of histopague 1077 (Sigma Diagnostics, St. Louis, Mo.) were slowly placed underneath the cells suspended in 7 ml of PBS, in a 50-ml tube. The tube was centrifuged at 1,640 rpm for 15 minutes at room temperature. Cells at the interface were collected, diluted in a 50-ml tube, and washed twice with ice-cold PBS (1,250 rpm for 10 minutes). Approximately 1×10⁶ cells/mouse liver were recovered. The viability by trypan blue staining was more than 95%. Both splenocytes and liver-associated lymphocytes were isolated from all animals in all experimental groups.

Flow Cytometry Analysis for Determination of NK1.1⁺Lymphocyte Depletion

Immediately following lymphocyte isolation, triplicates of 2-5×10⁴ cells/500 μl PBS were put into Falcon 2052 tubes incubated with 4 ml of 1% BSA for 10 minutes, and centrifuged at 1400 rpm for 5 minutes. Cells were resuspended in 10 μl FCS with 1:20 FITC-anti mouse NK1.1 antibody (NKR-P1C, Pharmingen, USA), and mixed every 10 minutes for 30 minutes. Cells were washed twice in 1% BSA, and kept in 4° C. until reading. For the control group, only 5 μl of 1% BSA was added. Analytical cell sorting was performed on 1×10⁴ cells from each group with a fluorescence-activated cell sorter (FACSTAR plus, Becton Dickinson). Only live cells were counted, and background fluorescence from non-antibody-treated lymphocytes were deducted from the levels obtained. Gates were set on forward- and side-scatters to exclude dead cells and red blood cells. The data were analyzed with Consort 30 two-color contour plot program (Becton Dickinson, Oxnard, Calif.), or the CELLQuest program.

Splenocyte and Liver-associated-lymphocyte Cultures

Splenocytes and liver-associated-lymphocytes were harvested from mice in all groups (A′ to F′) and cultured in 24 well tissue culture plates. Triplicates were prepared from each animal in all study groups and cultured for 12 hours. Lymphocytes were activated in cell dishes 1×10⁶ splenocytes/ml RPMI 1640 with Con A 2 μg/ml and 2 μM monensin (Biosource, Calif.) required to prevent cytokines from being released from cells for 12 h at 37° C. in 5%. The RPMI medium contains: 10% FCS, 200 mM Hepes, 100 U of penicillin and 100 Mg of streptomycin/ml, 10 mM Hepes IL2-10 U/ml, CEP-50 Mg/ml. Cells included 2.5×10⁶ splenocytes and 0.5×10⁶ LAL, with Monensin 2 μM (Biosource, Calif.). Supernatant fluids were collected from both sets for cytokine measurements by ELISA, and lymphocytes were analyzed by flow cytometry as described [Collins, C., et al., Eur. J. Immunol. 26:3114-3118 (1996)].

Intracellular Staining and Flow Cytometry

Cells were harvested from all wells and double stained. Extracellular and intracellular staining to detect CD4⁺ T-cell populations (Th1 and Th2 cells) were used as previously described using the following antibodies: FITC conjugated anti CD4, and PE-conjugated anti IL4 mAb were used for detection of CD4+IL4+ cells (PharMingen, San Diego, Calif.). FITC conjugated anti CD4 and PE-conjugated anti IFNγ mAb were used for detection of CD4+IFNγ cells (PharMingen, San Diego, Calif.). All was done according to the manufacturer's instructions (IC screen, Biosource intracellular staining kit, Calif.). Lymphocytes were analyzed by flow cytometry.

Liver Lymphocyte Cytotoxicity Assays

The target cells used in these studies were YAC-1 cells, a lymphoma cell line adapted to continuous growth in tissue culture by employing supplemented RPMI with 10% FCS. YAC-1 cells were prepared for NK assay by seeding them at a density of 2×10⁵ cells/ml in 25 ml flasks with RPMI 10% FCS, and collecting them 24 hours later. Cells were suspended and collected in a 50 ml tube and washed twice with medium by centrifugation (1250 rpm) for 10 minutes. This procedure ensured efficient labeling with ⁵¹Cr and high sensitivity of lysis by NK cells. Target cells were labeled with ⁵¹Cr (New Life Science, Boston Mass., Gamidor, Israel) and incubated for 90 minutes at 37° C. (200 mCi/2×10⁶ cells in 300 μl RPMI medium). Cells were manually mixed every 10 minutes. Following incubation, 3 ml of 20% FCS RPMI were added, and reincubated for 30 minutes at 37° C. Cells were washed three times in RPMI 10% FCS and counted. For determination of degree of labeling efficiency, 100 μl of cells were counted, and a minimum of 0.6 cpm/cell were measured. Effector cells were liver lymphocytes isolated from livers from groups A-H described above. The ⁵¹Cr-release assay was performed in Costar 96-well plates. A graded number of effector cells in 100 μl were mixed with 5000 labeled target cells in 100 μl, with effector to target ratios (E:T ratio) of 100:1, 50:1, and 10:1. Each well contained target and effector cells in a total volume of 200 μl. Five wells were tested for each ratio from each sample. For determination of spontaneous release, 6 wells of a similar number of target cells were plated with 100 μl RPMI 10% FCS. For determination of maximum release, 6 wells of target cells in 100 μl medium were mixed with 100 μl TritonX. The plate was centrifuged for 2 minutes (500 rpm) followed by 4 hours of incubation in 5% CO2 at 37° C. The plate was than centrifuged again for 2 minutes (500 rpm), and supernatants were harvested and counted using a gamma counter. Results were expressed as percent specific lysis of target cells calculated by using the equation: % cytotoxicity=mean cpm of assay-cpm from spontaneous release/cpm from targets lysed with TritonX-cpm from spontaneous release ×100.

Cytokine Secretion

Supernatant fluids were collected from both sets of triplicates and cytokine levels were measured for all mice from all tolerized and non-tolerized groups, NK1.1 depleted and non-deleted mice. IL4, IL10, IL12, and IFNγ levels were measured by a “sandwich” ELISA, using Genzyme Diagnostics kits (Genzyme Diagnostics, Mass., USA) according to the manufacturer's instructions. Serum levels were measured in 5 mice from tolerized and non-tolerized NK1.1 depleted and non-depleted mice, 10 days after colitis induction.

In vitro Education Experiments

Isolation and Separation of Lymphocytes

Splenocytes were prepared and separated into four subsets of lymphocytes, CD4⁺, CD8⁺, NK, and Dendritic cells. Cell separation was done using Magnetic Cell Sorting (MACS). Specific microbeads were used for each subset of lymphocytes: CD4 and CD8 microbeads, and anti-NK beads (Miltenyl Biotec, Germany). Immediately following lymphocyte isolation, triplicates of 2-5×10⁴ cells/500□l PBS were put into Falcon 2052 tubes incubated with 4 ml of 1% BSA for 10 minutes, and centrifuged at 1400 rpm for 5 minutes. Cells were re-suspended in 10 μl FCS with 1:20 FITC-anti mouse NK1.1 antibody (NKR-P1C, Pharmingen, USA), and mixed every 10 minutes for 30 minutes. Cells were washed twice in 1% BSA, and kept in 4° C. until reading. For the control group, only 5 μl of 1% BSA was added. Analytical cell sorting was performed on 1×10⁴ cells from each group with a fluorescence-activated cell sorter (FACSTAR plus, Becton Dickinson). Only live cells were counted, and background fluorescence from non-antibody-treated lymphocytes was deducted from the levels obtained. Gates were set on forward- and side-scatters to exclude dead cells and red blood cells. The data was analyzed with the Consort 30 two-color contour plot program (Becton Dickinson, Oxnard, Calif.), or the CELLQuest program.

Splenocyte and Liver-associated-lymphocyte Cultures

Splenocytes were harvested from mice in all groups and cultured in 24 well tissue culture plates. Triplicates were prepared from each animal in all study groups and cultured for 12 hours. Supernatant fluids were collected from both sets for cytokine measurements by ELISA.

### Example 1

The Effect of Tolerance Induction in Experimental Colitis

To evaluate the effect of tolerance induction in the experimental colitis model, six groups of mice, consisting of 20 animals each, were studied (Table 1). All mice were challenged with rectal TNBS (groups A, B, D, and E), or with normal saline (control groups C and F) on day 1 of the study. Mice in all groups were fed (50 μg/mouse) every other day for 11 days beginning with the day of colitis induction. Groups B and E included mice fed with colitis extracted proteins (CEP). Mice in groups A, C, D, and F, were fed with bovine serum albumin (BSA, 50 μg/mouse). Mice in all groups were sacrificed 14 days following colitis induction. Mice in groups D to F were treated with anti-NK1.1 anti-mouse monoclonal antibodies 36 hours before termination of the study, as described above. Mice in groups A to C were not NK1.1-depleted.

TABLE 1
Experimental and control groups

| Group | NK1.1 depletion | Antigen fed | Rectal challenge |
|---|---|---|---|
| A | − | BSA | TNBS |
| B | − | CEP | TNBS |
| C | − | BSA | NS |
| D | + | BSA | TNBS |
| E | + | CEP | TNBS |
| F | + | BSA | NS |

BSA: Bovine serum albumin
CEP: colitis extracted protein
TNBS: 2,4,6,-trinitrobenzene sulfonic acid

Clinical Assessment of Colitis

A marked decrease in diarrhea was observed in tolerized mice from groups B and D fed with mouse-CEP or NK1.1-depleted respectively. In contrast, mice from groups A and E, fed with BSA or fed with mouse-CEP and NK1.1-depleted, suffered severe diarrhea. A follow up of mice body weight disclosed a statistically significant increase in body weight among tolerized mice in groups B and D, as compared with mice in groups A and E (13.5% and 11.65% vs. 3.2% and 4.8%, respectively, p<0.005).

Macroscopic Grading of Colitis

Induction of oral tolerance by the feeding of mouse extracted colitis-derived proteins or NK1.1-depletion (groups B and D), markedly alleviated the macroscopic grading of colitis. The scores for tested macroscopic parameters of colitis were: degree of colonic ulceration, intestinal and peritoneal adhesions, wall thickness, and degree of mucosal edema. The total macroscopic score was 0.35±0.01 and 0.63±0.03 in groups B and D mice respectively, compared with 3.1±0.54 and 3.05±0.67 in the non-treated control and CEP-fed-NK1.1-depleted groups A and E respectively (p<0.005).

Grading of Histological Lesions

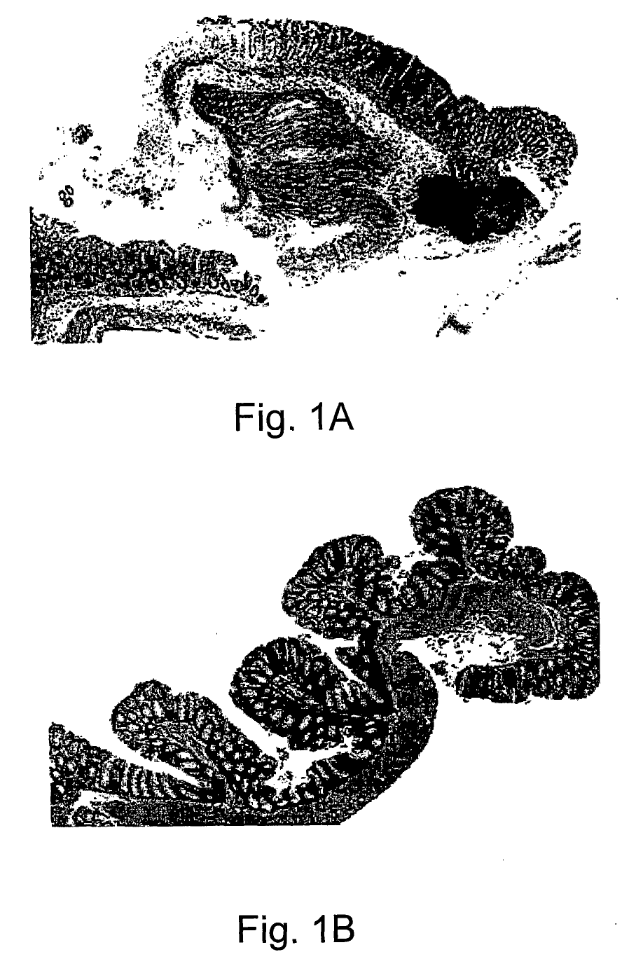

Histologic evaluation of bowel tissue showed a marked reduction in inflammatory response and mucosal ulcerations in tolerized or NK1. 1-depleted mice in groups B and D, as compared with non-tolerized mice in groups A and E. In mice in groups B and D, almost normal sections, or only minimal lymphocytic infiltration, was detected. In contrast, severe inflammatory reaction (grade 3-4) was observed in bowel specimens taken from non-tolerized mice (FIG. 1).

### Example 2

NK1.1⁺ Lymphocytes Increase the CD4+IL4+/CD4+IFNγ+ Ratio in Tolerized Mice and Decreased the CD4+IL4+/CD4+IFNγ+ Ratio in Non-tolerized Mice with Experimental Colitis

Tolerized Mice

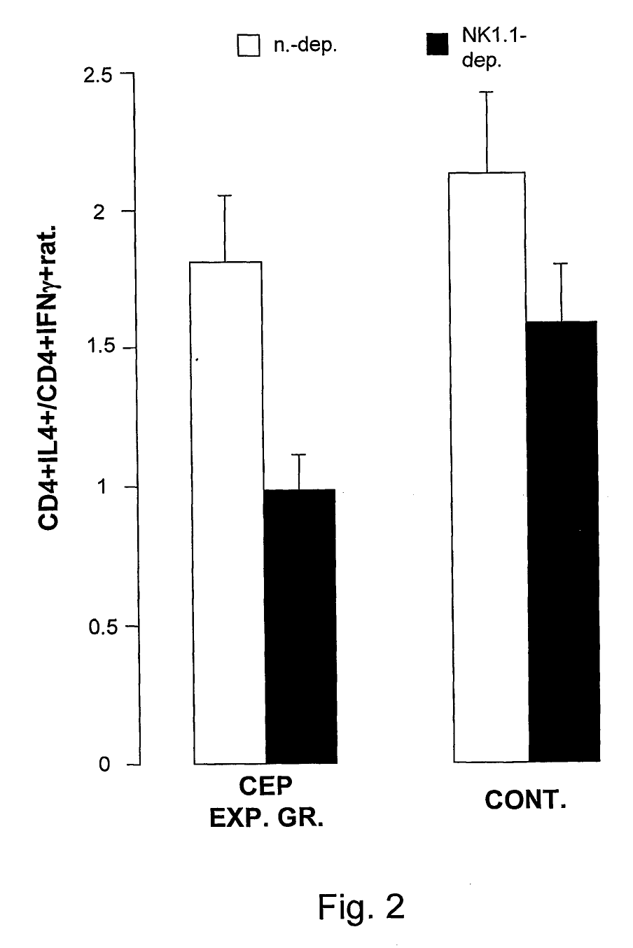

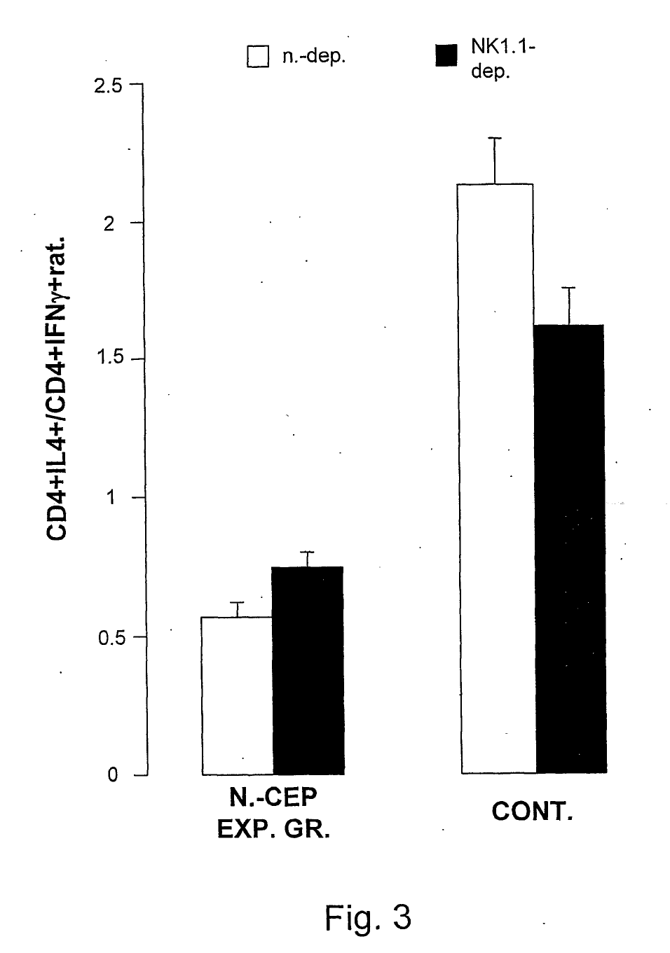

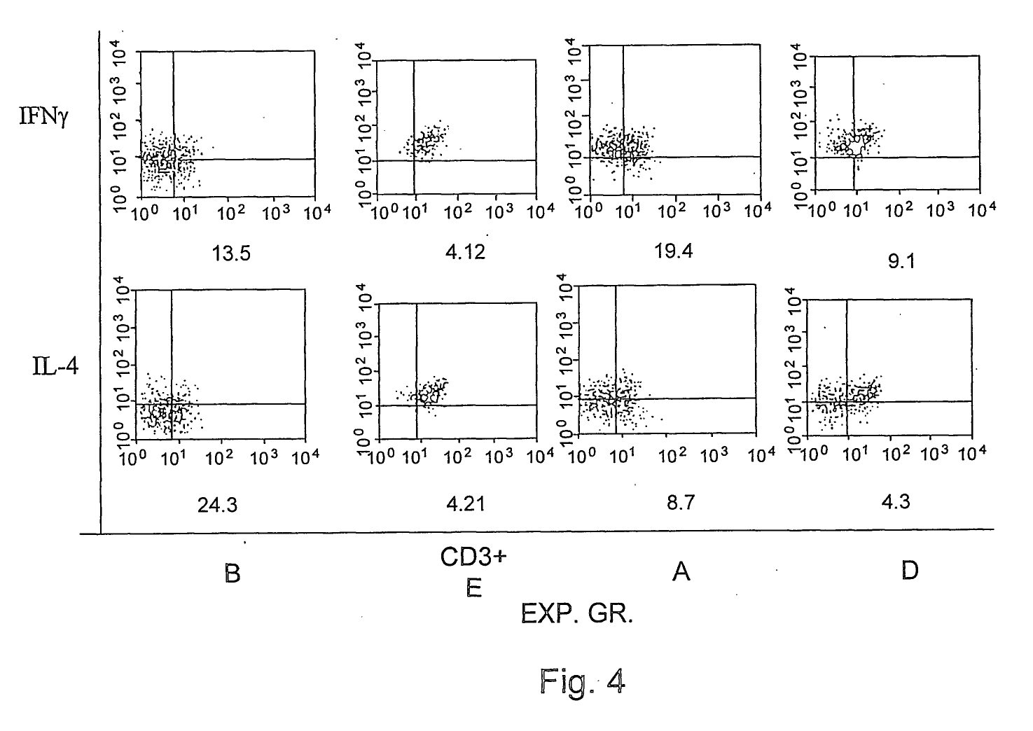

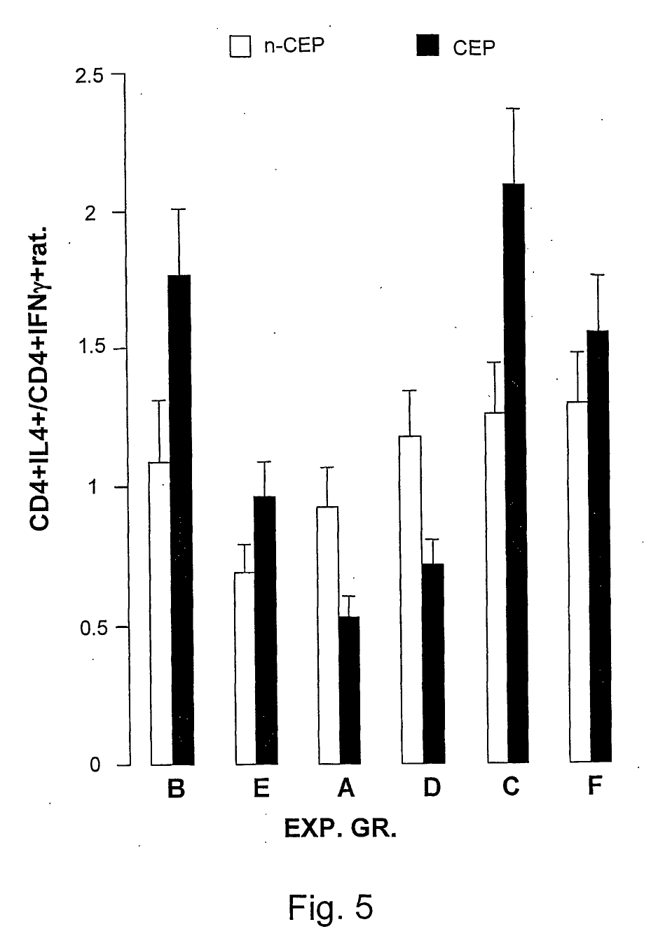

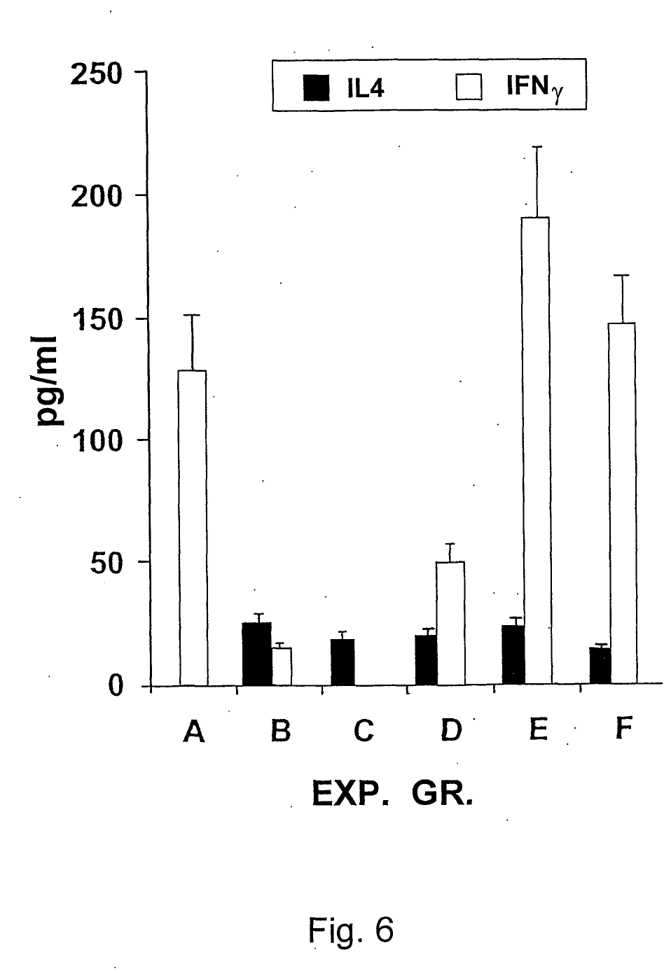

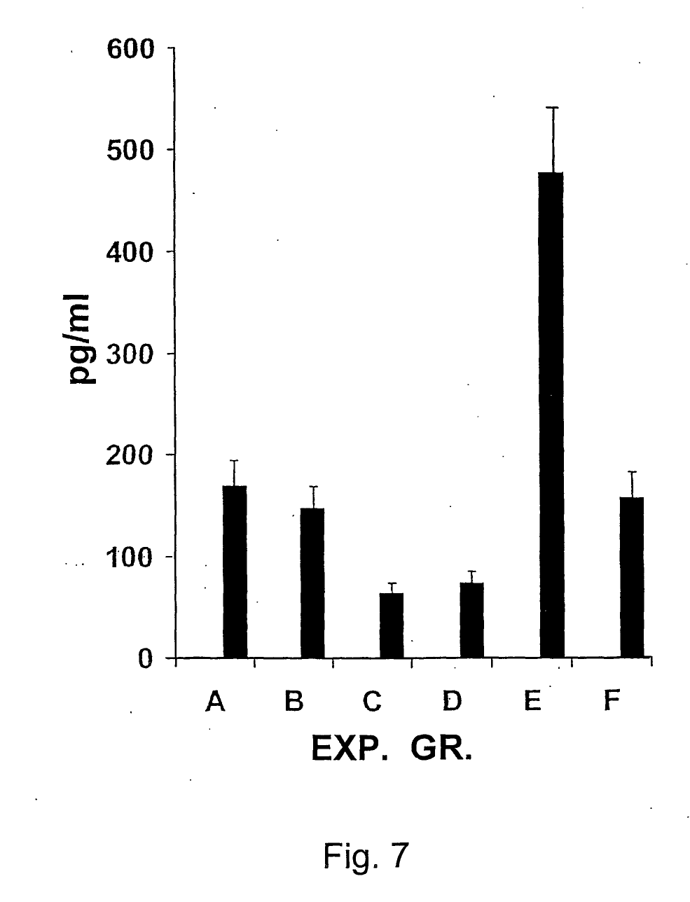

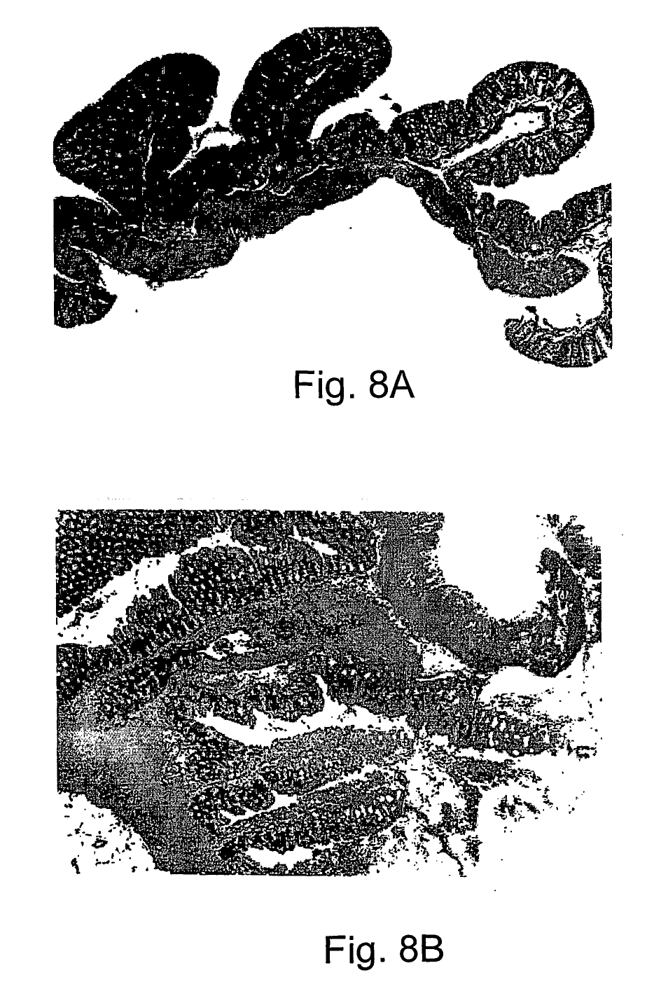

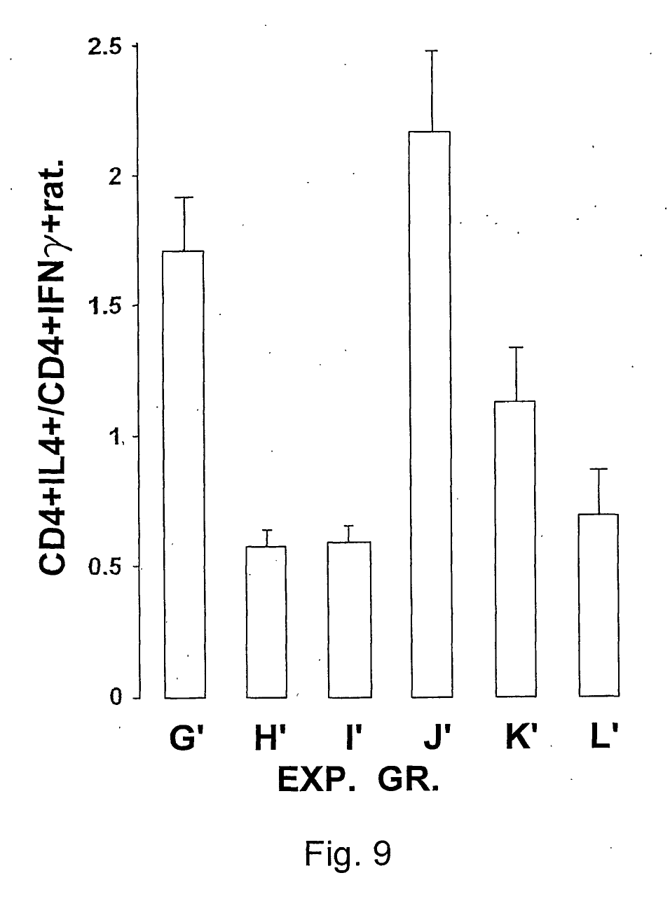

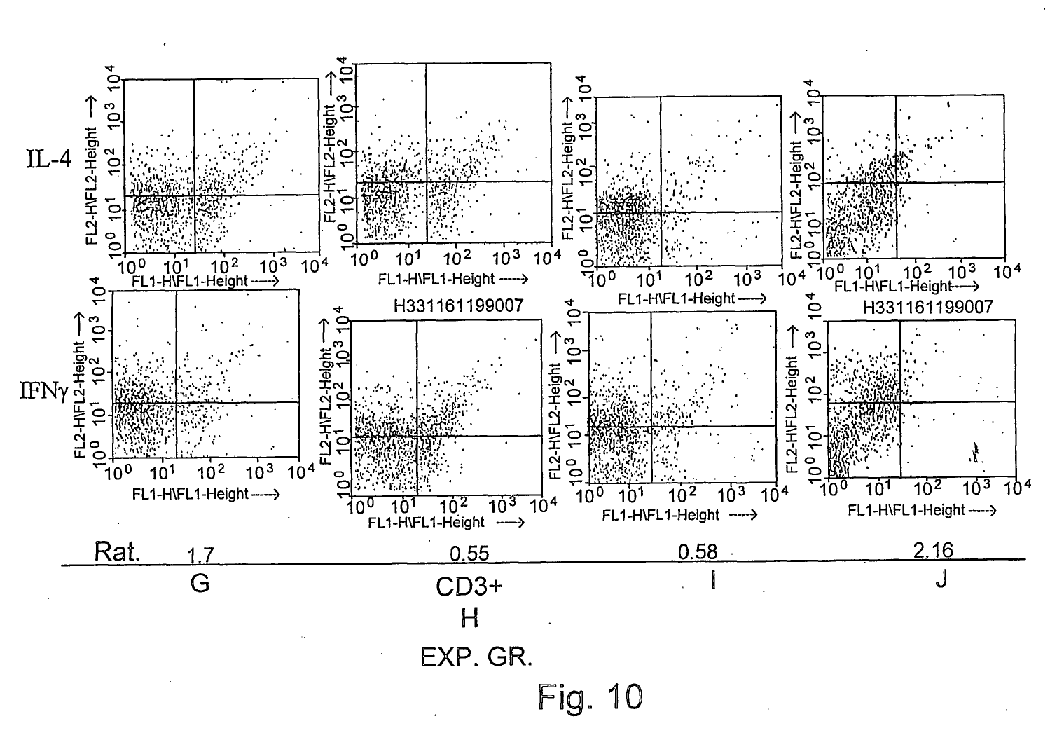

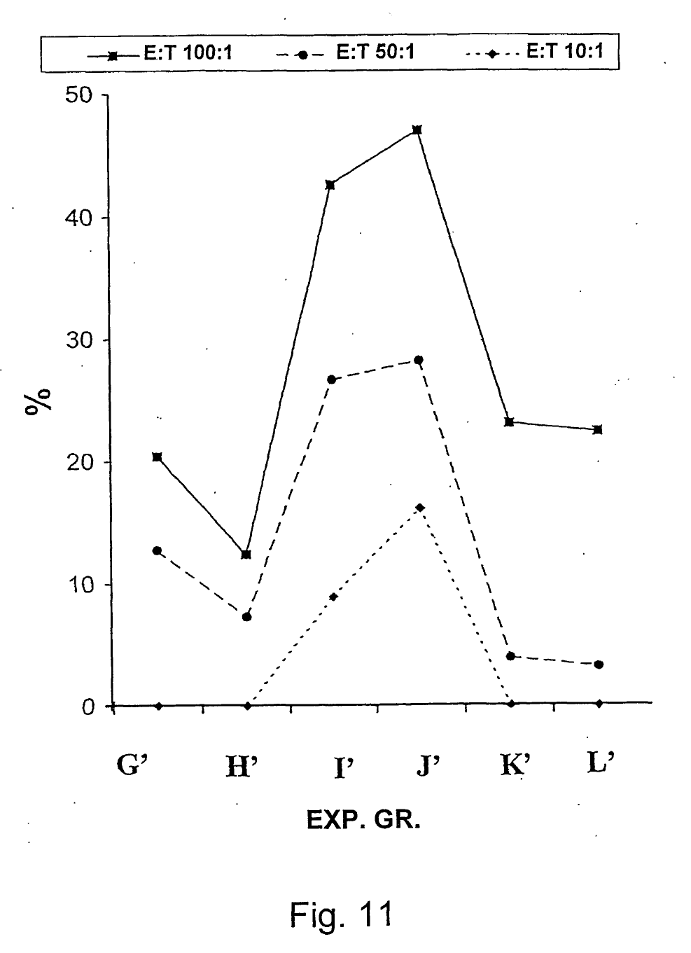

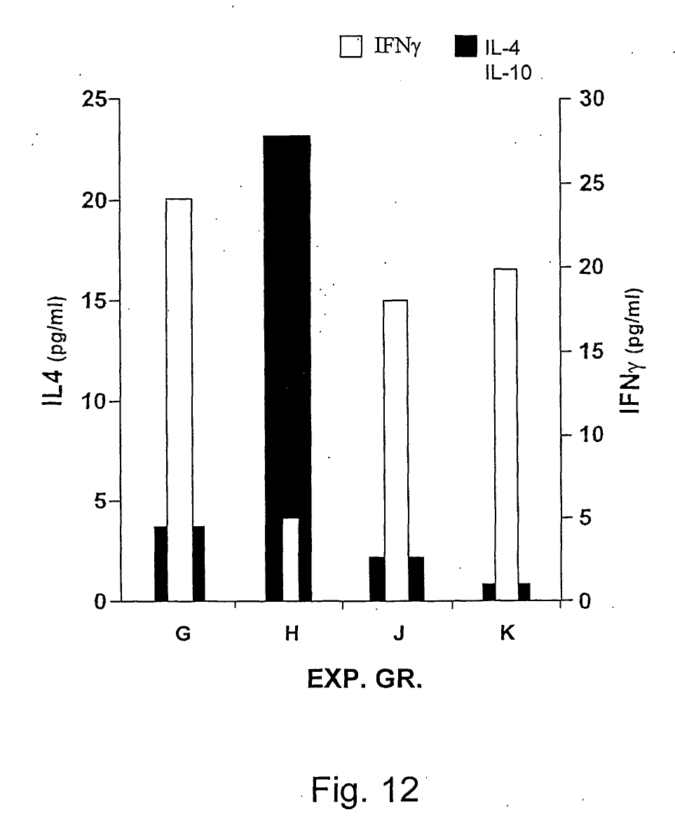

To study the effect of NK1.1+ lymphocytes in tolerized mice, splenocytes and liver-associated-lymphocytes (2.5×10⁶ splenocytes and 0.5×10⁶ LAL) were harvested from mice in all groups and cultured for 72 hours in the presence of CEP and APC. Flow cytometry analysis have shown that NK1.1-depletion following oral tolerance induction decreased the CD4+IL4+/CD4+IFNγ+ ratio in comparison with the non-NK1.1 LAL depleted tolerized mice (0.99±0.03 vs. 1.8±0.35 CD4+IL4+/CD4+IFNγ+, in groups E and B respectively, p<0.005, FIG. 2). The control NK1.1-depleted group (group F) disclosed a decrease in CD4+IL4+/CD4+IFNγ+ ratio compared with non-NK1.1-depleted group C (2.13±0.36 vs. 1.6±0.29, for groups C and F respectively).

Non-tolerized Mice

In contrast to tolerized groups, NK1.1-depletion had an opposite effect on non-tolerized mice with experimental colitis The CD4⁺IL4/CD4⁺IFNγ ratio increased in NK1.1-depleted non-tolerized groups, as compared with the non-NK1.1 depleted non-tolerized group (0.74±0.06 vs. 0.56±0.05 in groups A and D respectively, p<0.005, FIG. 3).

A comparison of the CD4+IL4+/CD4+IFNγ+ ratio between tolerized and non-tolerized mice revealed a higher ratio in all tolerized groups. Mice treated with TNBS and orally fed with CEP (group B) showed a significantly higher ratio, as compared with non-tolerized mice fed with BSA (group A). CD4+IL4+/CD4+IFNγ+ ratio in groups A, B, and C were: 0.56±0.05, 1.8±0.35 and 2.13±0.36 respectively (p<0.005). FIG. 4 shows the representative results of expression of IL4 and IFNγ on isolated lymphocytes from tolerized NK1.1 non-depleted and depleted mice from groups B and E, and non-tolerized NK1.1 non-depleted and depleted mice from groups A and D, respectively.

### Example 3

The Role of in-vitro Sensitization and the Effect of Disease-target-antigen on CD4+IL4+/CD4+IFNγ+ Ratio in Tolerized and Non-tolerized Mice with Experimental Colitis

For evaluation of the effect of in vitro exposure to the disease-target antigen on the CD4+IL4+/CD4+IFNγ+ ratio splenocytes and liver-associated-lymphocytes (2.5×10⁶) splenocytes and (0.5×10⁶) LAL were harvested from mice in all groups (listed in Table 1), and cultured for 12 hours, in the presence of Con A and in the absence of CEP and APC. An evaluation of the effect of NK1.1 depletion in the absence of antigen was similar to that found in the presence of antigen. Lymphocytes harvested from tolerized mice in group B revealed a significantly higher CD4+IL4+/CD4+IFNγ+ ratio, as compared with NK1.1-depleted mice in tolerized group E (0.7±0.02 vs. 1.1±0.02, respectively, p<0.005). In contrast, NK1.1 depletion induced an increase in the CD4+IL4+/CD4+IFNγ+ ratio in non-tolerized mice from groups A and D in the absence of antigen (1.21±0.03 vs. 0.96±0.01, respectively, p<0.005, Table 2, FIG. 5). These results suggest that immune education was achieved in vivo and was not affected by cell-antigen incubation in vitro.

Similarly, flow cytometry analysis has shown that the CD4+IL4+/CD4+IFNγ+ ratio decreased significantly in tolerized mice in groups B and E and in control groups C and F, and increased significantly in non-tolerized mice in groups A and D (p<0.005, FIG. 5).

TABLE 2
Effect of NK1.1 depletion and of disease-target-antigen on
CD4 + IL4 + /CD4 + IFNγ + ratio

| | | | CD4 + IL4 + / | CD4 + IL4 + / |
|---|---|---|---|---|
| | | | CD4 + IFNγ + | CD4 + IFNγ + |
| | | NK1.1 | (with tolerizing | (without tolerizing |
| Group | Tolerized | Depletion | antigen) | antigen) |
| A | − | − | 0.56 ± 0.05 | 0.96 ± 0.01 |
| B | + | − | 1.8 ± 0.35 | 1.1 ± 0.02 |
| C | Naive | − | 2.13 ± 0.36 | 1.3 ± 0.21 |
| D | − | + | 0.74 ± 0.06 | 1.21 ± 0.03 |
| E | + | + | 0.99 ± 0.03 | 0.7 ± 0.02 |
| F | Naive | + | 1.6 ± 0.29 | 1.33 ± 0.27 |

Change in Cytokine Levels in Tolerized and Non-tolerized Mice

Supernatant fluids were collected from both sets of triplicates and cytokine levels were measured for all mice from all tolerized and non-tolerized groups. IL4, and IFNγ levels were measured by a “sandwich” ELISA. Tolerized mice manifested a shift from Th1 to Th2 immune response cytokine secretion. These mice (group B) manifested an increase in IL4 levels and a decrease in IFNγ levels. In contrast, mice from non-tolerized groups (groups A, E) exhibited high IFNγ and low IL4 levels. Lymphocytes harvested from tolerized mice in group B revealed significantly higher IL4, and lower IFNγ levels, as compared with NK1.1-depleted mice in tolerized group E (24.4±1.4 and 14.1±0.4 vs. 22.6±0.7 and 189.8±8.4, respectively, FIG. 6). In contrast, NK1.1 depletion induced an increase in IFNγ and a decrease in IL4 levels in non-tolerized mice from groups A and D, in the absence of antigen (128.3±3.7 and 0.6±0.01 vs. 48.3±4.1 and 19.1±0.4, respectively, FIG. 6). NK1.1 depletion led to an increase in IL12 levels in the CEP-fed groups (475±23.3 vs. 145±5.7 and, for groups E, respectively, FIG. 7) but had an opposite effect in the non-CEP fed groups (165±7.4 and 74±3.3, for groups A and D respectively).

### Example 4

The Effect of Tolerance Induction on Adoptive Transfer of Splenocytes in Experimental Colitis

To evaluate the effect of tolerance induction in the experimental colitis model, six groups of donor mice consisting of 10 animals each were studied (the different groups are listed in Table 3). Colitis was induced in mice from groups G to J by rectal challenge with TNBS. Control mice in groups K and L were challenged with normal saline. Mice in all groups were fed with 50 μg/mouse every other day for 11 days starting on day of colitis induction. Groups I and J included mice fed with colitis extracted protein (CEP). Mice in groups G, H, K and L were fed with bovine serum albumin (BSA 50 μg/mouse). NK1.1 depletion was performed as described above in mice from groups G, I and K 36 hours prior to splenocyte harvesting. Mice in all groups were sacrificed 14 days following colitis induction.

The recipient mice groups G′-L′, consisting of 10 animals each were studied as well. Recipient mice were sublethally irradiated with 300 rad total body irradiation, 24 hours before intravenous injection of 1×10⁶ donor cells in 0.5 ml PBS. All mice were treated with TNBS enemas, 24 hours following cell transplantation. Clinical, macroscopic and histological parameters for colitis were determined 14 days following colitis induction as described below.

TABLE 3
Experimental and control groups

| | | | | SPLENO- |
|---|---|---|---|---|
| | NK1.1 | ANTIGEN | RECTAL | CYTE |
| GROUP | DEPLETION | FED | CHALLENGE | DONORS |
| DONORS: | | | | |
| G | + | BSA | TNBS | — |
| H | − | BSA | TNBS | — |
| I | + | CEP | TNBS | — |
| J | − | CEP | TNBS | — |
| K | + | BSA | NS | — |
| L | − | BSA | NS | — |
| G′ | − | — | TNBS | G |
| H′ | − | — | TNBS | H |
| I′ | − | — | TNBS | I |
| J′ | − | — | TNBS | J |
| K′ | − | — | TNBS | K |
| L′ | − | — | TNBS | L |

BSA: Bovine serum albumin
CEP: colitis extracted protein
TNBS: 2,4,6,-trinitrobenzene sulfonic acid

Clinical Assessment of Colitis

A marked decrease in diarrhea was observed in recipients of tolerized cells from tolerized mice from group J′ fed with mouse CEP, as well as in the tolerized mice of group J. In contrast recipients of non-tolerized splenocytes from group H′ and mice fed with BSA from group H, suffered severe diarrhea. Follow up of mice body weight disclosed a statistically significant increase in body weights among tolerized mice in groups J and J′ compared with non-tolerized mice in groups H and H′ (10.8% and 11.2% vs. 5.7 and 5.5%, respectively, p<0.005).

Recipients of splenocytes from NK1.1 depleted-mice from group G′ and their donors from group G suffered less diarrhea compared with non-tolerized mice in groups H and H′. Mice from both groups (G and G′) showed increase in body weights (9.9% and 10.2% vs.5.7% and 5.5% respectively, p<0.005). In contrast, recipients of splenocytes from NK1.1-depleted mice from group I′ led to loss of the tolerizing effect. A similar effect was observed in their donors (group I). These mice disclosed less diarrhea when compared with groups H and H′, however were worse than non-NK1.1 depleted controls. Similarly, no significant increase in body weights was observed in mice in both groups (6.0% and 5.1%, for mice in groups I and I′, respectively, p<0.005, compared with tolerized mice in groups J and J′).

Mice from groups K and L were not challenged with TNBS and did not show clinical evidence of disease. Their body weights increased by 11.4% and 12.3% respectively. In contrast, mice from groups K′ and L′ developed severe diarrhea and their body weights increased only by 4.5% and 5.2% respectively.

Macroscopic Grading of Colitis

Induction of oral tolerance by the feeding of mouse extracted colitis-derived proteins (group J), and adoptive transfer of tolerized lymphocytes (group J′) markedly alleviated the macroscopic grading of colitis. The scores for tested macroscopic parameters of colitis were: degree of colonic ulceration, intestinal and peritoneal adhesions, wall thickness, and degree of mucosal edema. The total macroscopic score was 0.31±0.24 and 0.3±0.25 in groups J and J′ respectively, compared with 3.22±0.15 and 3.32±0.26 in non-tolerized mice in groups H and H′, respectively. NK1.1 depleted mice from group G and recipients of their lymphocytes from group G′ manifested alleviation of disease (0.8±0.4 and 0.85±0.5 respectively). In contrast, NK1.1-depleted mice from group I and recipients of their lymphocytes (group I′) manifested severe colitis (3.72±0.22 and 3.77±0.6 respectively, p<0.005). Mice form groups K′ and L′ showed evidence of severe colitis (3.4±0.29 and 3.27±0.22, respectively).

Grading of Histological Lesions

Histologic evaluation of bowel tissue showed a marked reduction in inflammatory response and mucosal ulcerations in tolerized mice in groups J and J′, with histological scores of 1.8 and 1.7 respectively. In these mice almost normal sections, or only minimal lymphocytic infiltration, was detected. In contrast, severe inflammatory reaction was observed in bowel specimens taken from non-tolerized mice in groups H and H′ with histological scores of 3.3 and 3.08 (groups H and H′, respectively, p<0.005, FIG. 8). A marked reduction in inflammatory response and mucosal ulcerations was detected in non-tolerized NK1.1-depleted mice in group G and recipients of their splenocytes (group G′). The histological scores for groups G and G′ were 2.08 and 2 respectively. NK1.1-depleted mice from group I and recipients of their lymphocytes (group I′) manifested severe colitis. Scores for mice in groups I and I′ were 2.9 and 2.5 respectively. Groups K and L were not rectal challenge with TNBS. Mice form groups K′ and L′ showed evidence of severe colitis with scores of 3.1 and 3, respectively.

### Example 5

NK1.1+ Lymphocytes Increase the CD4+IL4+/CD4+IFNγ+ Ratio in Tolerized Mice and Decreased the CD4+IL4+/CD4+IFNγ+ Ratio in Non-tolerized Mice with Experimental Colitis.

Tolerized Mice

A comparison of the CD4+IL4+/CD4+IFNγ+ ratio between tolerized and non-tolerized recipient mice revealed a higher ratio in all tolerized groups. Tolerized recipient mice from group J′ showed a significantly higher ratio, as compared with non-tolerized mice in group H′. CD4+IL4+/CD4+IFNγ+ ratio were: 2.16 and 0.55 respectively (p<0.005).

Adoptive transfer of lymphocytes from tolerized mice increased the CD4+IL4/CD4+IFNγ+ ratio in recipients mice. Flow cytometry analysis have shown that adoptive transfer of splenocytes from NK1.1-depleted CEP fed donor mice decreased the CD4+IL4/CD4+IFNγ+ ratio, compared to splenocytes harvested from tolerized non-depleted mice (0.58 vs. 2.16, for groups I′ and J′ respectively, p<0.005, FIG. 9).

Non-tolerized Mice

Adoptive transfer of non-tolerized lymphocytes decreased the CD4+IL4/CD4+IFNγ+ ratio in recipient mice. In contrast to tolerized groups, NK1.1-depletion had an opposite effect on non-tolerized mice with experimental colitis. Flow cytometry analyses have shown that adoptive transfer of splenocytes from NK1.1-depleted non-tolerized donor mice increased the CD4+IL4/CD4+IFNγ+ ratio compared with splenocytes from non-tolerized non-NK1.1 depleted mice. (1.7 vs. 0.55, in groups G′ and H′, respectively, p<0.005, FIG. 9 and Table 4). FIG. 10 shows representative results of expression of IL4 and IFNγ on isolated lymphocytes from recipients of tolerized NK1.1 non-depleted and depleted donors, and from recipients from non-tolerized NK1.1 non-depleted and depleted donors (groups G′-J′).

TABLE 4
Effect of adoptive transfer of tolerized and non-tolerized,
NK1.1-depleted and non-depleted splenocytes
CD4 + IL4 + /CD4 + IFNγ + ratio

| Recipients: | Donors: | CD4 + IL4 + /CD4 + IFNγ + ratio |
|---|---|---|
| G′ | G | 1.7 |
| H′ | H | 0.55 |
| I′ | I | 0.58 |
| J′ | J | 2.16 |
| K′ | K | 1.13 |
| L′ | L | 0.69 |

Adoptive Transfer from Control Lymphocytes

Flow cytometry analyses have shown that adoptive transfer of splenocytes from control NK1.1-depleted mice increased the CD4+IL4/CD4+IFNγ+ ratio compared to non NK1.1-depleted donor mice. (1.13 vs. 0.69, groups K′ and L′ respectively, p<0.005).

### Example 6

Liver Lymphocytes Cytotoxicity by NK1.1

YAC-1 cells were used as target cells in these studies at an E:T ratio of 100:1, 50:1 lysis and 10:1. Studies were performed using liver lymphocytes isolated from recipients of NK1.1 depleted and non-depleted tolerized and non-tolerized mice. Recipients from non-tolerized non-NK1.1 depleted mice (group H′) showed almost no lysis compared to the other groups 12.37% cytotoxicity (100:1 E: T, FIG. 11). Recipients from non-tolerized NK1.1-depleted mice in group G′ showed higher lysis than group H′ 20.4% vs. 12.37% of cytotoxicity, respectively. Recipients from NK1.1-depleted CEP fed mice from group I′ showed lower lysis than non NK1.1 depleted mice in group J′ (42.58% vs. 46.98% cytotoxicity, respectively). Recipients from control groups had 23.1% vs. 22.47% cytotoxicity, for mice in group K′ compared with Group L′ respectively (p<0.005, FIG. 11).

Cytokine Assay

Supernatant fluids were collected from both sets of triplicates and cytokine levels were measured for all mice from all tolerized and non-tolerized groups. IL4, IL10, and IFNγ levels were measured by a “sandwich” ELISA. Tolerized mice manifested a shift from Th1 to Th2 immune response cytokine secretion. These mice (group H) manifested an increase in IL4, IL10 levels and a decrease in IFNγ levels. In contrast, mice from non-tolerized groups (groups G, J, K) exhibited high IFNγ and low IL10 levels. Lymphocytes harvested from tolerized mice in group H revealed significantly higher IL4, IL10, and lower IFNγ levels, as compared with NK1.1-depleted mice in tolerized group K (18.4±3.7, 23.1±2.9 and 5.1±0.4 vs. 2.9±0.6, 0.8±0.1 and 19.8±3.8, respectively, FIG. 12). In contrast, NK1.1 depletion induced an increase in IFNγ and a decrease in IL4, IL10 levels in non-tolerized mice from groups G and J, in the absence of antigen (24.3±3.7, 3.1±0.9, and 4.6±0.4 vs. 18.3±1.1, 3.2±0.1 and 2.1±0.4, respectively, FIG. 12).

### Example 7

Ex-vivo Immune Programming of NK T Lymphocytes

As have been shown by the preceding Examples, induction of oral tolerance by feeding of mouse extracted colitis-derived proteins markedly alleviated different symptoms of colitis (macroscopic grading of colitis, severe diarrhea, inflammatory response and mucosal ulcerations) as compared with non-tolerized mice.

Therefore, the present inventors have preformed the following experiment in order to determine the possibility of in vitro/ex-vivo immune programming of NK T cells by examining whether an ex-vivo education of cells, particularly NK cells, may ameliorate different colitis symptoms in animals suffering from induced colitis that were not subjected to any oral tolerance treatment.

Different cell subgroups in eight different combinations (CD4, CD8, splenocytes and Dendritic cells, as listed in Table 5) were prepared from each of the following six experimental groups:

1. Cells harvested from control animals without colitis and without treatment (oral tolerization). These cells were incubated ex-vivo with BSA.

2. Cells harvested from control animals with colitis and without treatment (oral tolerization). These cells were incubated ex-vivo with BSA.

3. Cells harvested from animals with colitis and with treatment via oral toerization. Cells were incubated in vitro with BSA.

4. Cells harvested from control animals without colitis and without treatment (oral tolerization). These cells were incubated ex-vivo with CEP.

5. Cells harvested from control animals with colitis and without treatment (oral tolerization). Cells were incubated ex-vivo with CEP.

6. Cells harvested from animals with colitis and with treatment (oral tolerization) via oral toerization. Cells were incubated ex-vivo with CEP.

TABLE 5
Different experimental subgroups of cell type and combination

| Different subgroups | Cell type or cell combination |
|---|---|
| Group A″ | CD4 cells |
| Group B″ | CD8 cells |
| Group C″ | splenocytes |
| Group D″ | Dendritic cells (DC) |
| Group E″ | NK T cells |
| Group F″ | NK T + CD4 |
| Group G″ | NK T + CD8 |
| Group H″ | NK T + DC |

It is to be noted that cells from experimental groups 1, 2 and 3 were incubated in vitro in the presence of BSA and therefore served as control, whereas cells of experimental groups 4, 5 and 6 were incubated in vitro in the presence of the antigen (CEP) and therefore served as test groups. ex-vivo education was examined by measuring secretion of IL10 (as compared to IFN□secretion) by the different treated cells.

It is to be appreciated that different cell types or cell combinations (subgroups A″ to H″) which were prepared from animals suffering from colitis that were not treated (oral tolerization) but were incubated in vitro in the presence of CEP (subgroups 5A″ to 5H″), are the main tested groups indicating the feasibility of ex-vivo education by incubation with antigen. As shown by Table 6, culturing NK1.1+ T cells in the presence of disease associated antigens (subgroup E″5) leads to cytokine pattern that is similar to that of tolerized cells as manifested by increase IL10 secretion.

A similar pattern was observed for culturing of CD4 cells and antigen (subgroup A″5). These results indicate successful ex-vivo education by exposing cells to antigen associated with the disease. However combining of more than one cell type in the presence of antigen diminished this desired effect, as NK T education by antigen was prevented by the addition of CD4, CD8, or DC (subgroups F″5, G″5 and H″5, respectively).

In addition to feasibility of ex-vivo education of NK T cells by incubation with antigen associated with the disease, the inventors have examined whether co-culturing of NK T cells with other cell types may result in the desired ex-vivo education as reflected by IL10 elevated secretion. As shown by Table 6, only combination of NK T cells and CD4 or CD8 cells that were obtained from tolerated mice resulted in IL10 elevated secretion (subgroups F3 and G3, respectively). NK T and CD4 cells obtained from tolerated mice combined with ex-vivo exposure to antigen had a similar effect (subgroup F6), whereas the antigen presence significantly reduced IL10 secretion when the NK T CD8 from tolerized mice, combination was examined (subgroup G6).

However, co-culturing of NK T cells with dendritic cells failed to induce IL10 secretion in any combination examined (subgroups H3 to H6).

Experimental and control groups

| | TNBS | ISOLATED | ANTIGEN | ANTIGEN | | |
|---|---|---|---|---|---|---|
| GROUP | COLITIS | LYMPHOCYTES | FED | IN PLATE | IFN□ | IL10 |
| A″1 | − | CD4 | BSA | BSA | 4000 | 1450 |
| A″2 | + | CD4 | BSA | BSA | 36 | 200 |
| A″3 | + | CD4 | CEP | BSA | 0 | 53 |
| A″4 | − | CD4 | BSA | CEP | o | 0 |
| A″5 | + | CD4 | BSA | CEP | 0 | 270 |
| A″6 | + | CD4 | CEP | CEP | 0 | 66 |
| B″1 | − | CD8 | BSA | BSA | 4000 | 1500 |
| B″2 | + | CD8 | BSA | BSA | 0 | 305 |
| B″3 | + | CD8 | CEP | BSA | 50 | 165 |
| B″4 | − | CD8 | BSA | CEP | 0 | 0 |
| B″5 | + | CD8 | BSA | CEP | 0 | 54 |
| B″6 | + | CD8 | CEP | CEP | 0 | 98 |
| C″1 | − | SPLENOCYTES | BSA | BSA | 0 | 0 |
| C″2 | + | SPLENOCYTES | BSA | BSA | 230 | 160 |
| C″3 | + | SPLENOCYTES | CEP | BSA | 0 | 306 |
| C″4 | − | SPLENOCYTES | BSA | CEP | 0 | 0 |
| C″5 | + | SPLENOCYTES | BSA | CEP | 0 | 34 |
| C″6 | + | SPLENOCYTES | CEP | CEP | 0 | 420 |
| D″1 | − | DC | BSA | BSA | 240 | 120 |
| D″2 | + | DC | BSA | BSA | 4000 | 720 |
| D″3 | + | DC | CEP | BSA | 4000 | 920 |
| D″4 | − | DC | BSA | CEP | 0 | 0 |
| D″5 | + | DC | BSA | CEP | 140 | 170 |
| D″6 | + | DC | CEP | CEP | 30 | 280 |
| E″1 | − | NK T | BSA | BSA | o | o |
| E″2 | + | NK T | BSA | BSA | o | 52 |
| E″3 | + | NK T | CEP | BSA | 0 | 230 |
| E″4 | − | NK T | BSA | CEP | 0 | 14 |
| E″5 | + | NK T | BSA | CEP | 38 | 340 |
| E″6 | + | NK T | CEP | CEP | 60 | |
| F″1 | − | NK T + CD4 | BSA | BSA | 0 | 15 |
| F″2 | + | NK T + CD4 | BSA | BSA | 150 | 0 |
| F″3 | + | NK T + CD4 | CEP | BSA | 0 | 360 |
| F″4 | − | NK T + CD4 | BSA | CEP | 29 | 28 |
| F″5 | + | NK T + CD4 | BSA | CEP | 0 | 0 |
| F″6 | + | NK T + CD4 | CEP | CEP | 0 | 300 |
| G″1 | − | NK T + CD8 | BSA | BSA | 18 | 98 |
| G″2 | + | NK T + CD8 | BSA | BSA | 0 | 12 |
| G″3 | + | NK T + CD8 | CEP | BSA | 0 | 350 |
| G″4 | − | NK T + CD8 | BSA | CEP | 0 | 0 |
| G″5 | + | NK T + CD8 | BSA | CEP | 0 | 0 |
| G″6 | + | NK T + CD8 | CEP | CEP | 0 | 19 |
| H″1 | − | NK T + DC | BSA | BSA | 0 | 100 |
| H″2 | + | NK T + DC | BSA | BSA | 4000 | 270 |
| H″3 | + | NK T + DC | CEP | BSA | 0 | 98 |
| H″4 | − | NK T + DC | BSA | CEP | 0 | 0 |
| H″5 | + | NK T + DC | BSA | CEP | 0 | 0 |
| H″6 | + | NK T + DC | CEP | CEP | 44 | 80 |

BSA: Bovine serum albumin
CEP: colitis extracted protein
TNBS: 2,4,6,-trinitrobenezene sulfonic acid
DC: Dendritic cells

The Examples of the present invention have shown that adoptive transfer of tolerized splenocytes into naive mice induced tolerance, since it is assumed that Th2 specific memory cells were transferred. In contrast adoptive transfer of lymphocytes from NK1.1 depleted CEP fed mice, failed to transfer the tolerance, and upregulated the inflammatory Th1 mediated response. It was found that NK1.1+ T cells rapidly produce IL4, and play a regulatory role in autoimmune response in the experimental allergic encephalomyelitis and in the diabetic NOD mice models [Bendelac, A., et al., Annu Rev Immunol 15: 535-562 (1997); Sakamoto, A., et al., J Allergy Clin Immunol 103(5 pt 2): s445-51(1999); Seki, S., et al., J Immunol 147:1214-1221 (1991)]. However depletion of NK1.1 T cells at termination of oral tolerance induction affected the type of cytokine secretion decreasing the CD4+IL4+/CD4+IFNγ ratio compared with tolerized non-depleted NK1.1 T cells mice. The results of the present invention suggest that NK1.1 T cells may influence the Th1/Th2 profile of immune responses via IFNγ pro-inflammatory or via IL4 anti-inflammatory cytokine secretion. In both conditions, their impact is far greater than that by conventional CD4+ T cells [Chen, H. et al., J Immunol 159:2240-2249 (1997)].

Furthermore, the present inventors have further showed that ex vivo education of NK T cells is feasible. Since exposure of NK T cells in vitro to the disease target antigen enabled education of these cells towards the anti-inflammtory IL10 secretion pattern.

In conclusion, NK1.1+ lymphocytes play a dual role in immune modulation and in switching the immune response in the immunogenic or tolerogenic directions. The environment in which they become activated, different types of stimulations, or signaling receptors may determine their function. It is noteworthy that NK1.1+ T cells which are involved in distinct immunoregulatory mechanisms, and modulate the type of effector cells and the Th1/Th2 paradigm in immune-mediated disorders.

In conclusion, NK1.1+ lymphocytes play a dual role in immune modulation and in switching the immune response in the immunogenic or tolerogenic directions. The environment in which they become activated, different types of stimulations, or signaling receptors may determine their function. It is noteworthy that NK1.1+ T cells which are involved in distinct immunoregulatory mechanisms, and modulate the type of effector cells and the Th1/Th2 paradigm in immune-mediated disorders.

II.

Materials and Methods

Animals

Female immunocompetent (heterozygous) and athymic Balb/C mice were purchased from Jackson Laboratories, Bar Harbor, Me. All animals were kept in laminar flow hoods in sterilized cages and given irradiated food and sterile acidified water. Animal experiments were carried out in accordance with the guidelines of the Hebrew University-Hadassah Institutional Committee for Care and Use of Laboratory Animals, and with the committee's approval.

Cell Cultures

The HBsAg secreting human hepatoma cell line Hep-3B was grown in culture as monolayers, in a medium supplemented with non-essential amino acids and 10% heat inactivated fetal bovine serum.

Tumor and Splenocyte Transplantation in Athymic Mice

Athymic mice were conditioned with sub-lethal irradiation (600 cGy). Twenty-four hours after irradiation, the mice were injected subcutaneously into the right shoulder with 10⁷ human hepatoma Hep3B cells. Three days following irradiation, splenocytes were harvested from donor immunocompetent mice. Recipient athymic mice were injected intravenously with donor spleen cells at 1 ×10⁷ cells/mouse, establishing a competent immune system in the recipient. The mice were subsequently injected with antigen pulsed NKT cells.

Isolation of Lymphocytes and Separation of NKT Cells

Splenocytes were prepared from spleens harvested from donor immune-competent Balb/C mice. After the removal of connective tissue from spleens, they were placed in a 10 ml dish in cold sterile PBS and crushed through a stainless steel mesh (size 60, Sigma Chemical Co., St. Louis, Mo.). For lymphocyte isolation, 20 ml of histopaque 1077 (Sigma Chemical Co., St. Louis, Mo.) was placed underneath the cells suspended in 7 ml of PBS, in a 50 ml tube. Cells at the interface were collected, diluted in a 50 ml tube and washed twice with ice-cold PBS (1250 rpm for 10 minutes). Approximately 1×10⁸ cells/mouse were recovered. NKT cell separation was done using Magnetic Cell Sorting (MACS) with specific anti-NK microbeads (Miltenyl Biotec, Germany).

NKT Cell Education by Pulsing

NKT cells were educated by ex vivo pulsing with tumor or viral-associated antigens. 1×10⁶ NKT cells in 0.5 ml of PBS were placed in flasks and incubated with HCC lysate (3 μg/ml), Hep3B cells or bovine serum albumin (BSA) (1 μg/ml), for 72 hours.

Adoptive Transfer of NKT Cells

Adoptive transfer was performed seven days after the restoration of immune competence. This was carried out by the intravenous injection of NKT cells that were exposed in vitro to HCC lysate, Hep3B cells or BSA.

Evaluation of the Effect of Adoptive Transfer of Ex-vivo Immune-Modulated Regulatory NKT Lymphocytes

The effect of adoptive transfer of ex-vivo educated NKT lymphocytes was evaluated by monitoring the following parameters:

Follow-up of Tumor Growth

Recipient mice were followed at bi-weekly intervals for six weeks. Survival, body weight and tumor volume (using calipers) were assessed. Mice that showed signs of distress and mice with excessive weight loss (more than 10% between measurements or more than 25% of initial body weight) were sacrificed.

Cytokine Secretion

During the fourth week, blood was drawn from mice in all groups and centrifuged at 14,000 rpm. Serum cytokine levels were measured by “sandwich” ELISA using Genzyme Diagnostics kits (Genzyme Diagnostics, MA, USA).

Isolation of Liver and Spleen Lymphocytes and Flow Cytometry Determination of CD4+, CD8+ and NKT Cells

Isolation of lymphocytes from liver and spleen was performed as described above and triplicates of 2×10⁴ cells/500 μl PBS were placed into Falcon 2052 tubes. The cells were resuspended in 10 μl fetal calf serum (FCS) with 1:20 FITC anti-mouse NK1.1 antibody (NKR-P1C, Pharmigen, U.S.A.) and mixed every ten minutes for thirty minutes. The cells were washed twice in 1% BSA and kept in 4° C. until reading. Analytical cell sorting was performed on 1×10⁴ cells from each group with a fluorescence activated cell sorter (FACStar^{plus}, Becton Dickinson, Calif.). Only live cells were counted, and background fluorescence from non-antibody treated lymphocytes was deducted from the levels obtained. Data was analyzed with Consort 30 two-color countour plot program (Beckton Dickinson, Calif.).

Western Blot Analysis for STAT 1-6

For the determination of STAT protein expression, splenocytes were obtained from mice in all groups. Tissue homogenates (200 mg/ml) were prepared in 0.25M sucrose/10 mM Tris-HCl, pH 7.4 using a glass homogenizer fitted with a motor-driven Teflon pestle. Proteins (100 μg/lane) were resolved by electrophoresis on SDS-polyacrylamide (7.5%) gels and electroblotted to nitrocellulose membranes, For the detection of the STAT proteins, the membranes were probed with a polyclonal rabbit anti-mice antibody directed at the different STAT proteins, followed by alkaline phosphatase-coupled goat anti-rabbit IgG (Bethyl Lab., Montgomery, Tex).

### Example 1

The Effect of Adoptive Transfer of Ex-vivo Immune-Modulated Regulatory NKT Lymphocytes

To evaluate the in vivo anti tumor effect of the adoptive transfer of educated NKT cells, four groups of athymic Balb/C mice (Groups A-D), consisting of 10 animals each, were studied (Table 1). All the mice were sublethally irradiated and transplanted with human Hep3B HCC. NKT cells prepared from immunocompetent Balb/C mice were pulsed ex-vivo with HCC-derived antigens (HCC lysate), Hep3B cells, and BSA. 1×10⁶ of educated NKT cells were subsequently injected into each HCC harboring mouse by adoptive transfer. Group A received NKT cells pulsed with HCC lysate; Group B received NKT cells pulsed with Hep3B cells; Group C received NKT cells pulsed with BSA; and Group D mice did not undergo NKT transplantation.

TABLE 1
Experimental and Control Groups

| | Transplanted | |
|---|---|---|
| Group | cells | Pulsing antigen |
| A | NKT | HCC lysate |
| B | NKT | Hep3B cells |
| C | NKT | BSA |
| D | None | HCC lysate |

To determine the mechanism of anti-tumor effect, intrasplenic lymphocyte populations were analyzed by FACS for NKT, CD4 and CD8 markers. Tumor size and weight, serum cytokine levels and splenocyte STAT 1-6 protein expression were also assessed.

Results

Adoptive transfer of NKT cells pulsed with HCC-derived antigens (Group A) resulted in the complete disappearance of tumors within four weeks, and attenuated weight loss (6.5%). In contrast, mice in Group B, Group C and Group D. developed large, necrotic tumors and severe weight loss (21%, 17%, and 23% weight loss in Group B, Group C and Group D, respectively, p<0.05); consequently, survival could not be assessed.

NKT/CD4 and CD8/CD4 ratios were significantly increased in Group A (12.3 vs. 6.4, 4.8 and 5.6 in Group B, Group C and Group D, for NKT/CD4 ratio, respectively, p<0.05). Expression of the transcription factor STAT4 was significantly increased in Group A, But not in Groups B-D. Serum levels of IFNγ were increased in Group A compared with Groups B, C and D (3.24, 1.77 and 1.38 timesfold, respectively, p<0.05)

Adoptive transfer of NKT lymphocytes pulsed ex-vivo with HCC-derived antigens lead to suppression of HCC in mice. NKT mediated anti-tumor activity was associated with enhanced Th1 immunity, manifested by increased anti-tumor NKT and CD8 lymphocyte numbers, increased expression of STAT 4, a marker for IL-2 activity, and elevated serum pro-inflammatory cytokine levels. Ex-vivo modulation of NKT lymphocytes holds promise as a novel mode of immune therapy for HCC.

1. A method for the treatment of immune-related disorders in a mammalian subject in need of such treatment, by manipulating NK T cell population of said subject, wherein manipulation of said NK T cell population results in modulation of the Th1/Th2 cell balance towards an inflammatory response, said modulation being mediated by different components of said subject's immune system.

2. A method for the treatment of immune-related disorders in a mammalian subject in need of such treatment, by manipulating NK T cell population of said subject, wherein manipulation of said NK T cell population results in modulation of the Th1/Th2 cell balance towards an anti-inflammatory or pro-inflammatory response, said modulation being mediated by different components of said subject's immune system.

3. A method for the treatment of immune-related disorders in a mammalian subject in need of such treatment, by manipulating NK T cell population of said subject, wherein manipulation of said NK T cell population results in modulation of the Th1/Th2 cell balance toward anti-inflammatory cytokine producing cells, said modulation being mediated by different components of said subject's immune system.

4. The method according to claims 1, 2 or 3, wherein said components are selected from the group consisting of cellular immune reaction elements, humoral immune reaction elements and cytokines.

5. The method according to claim 3, wherein said manipulation is performed by depletion of said NK T cell population.

6. A method for treatment of immune-related disorders in a mammalian subject according to claim 3, comprising the steps of:
a. obtaining NK T cells from said subject;
b. ex vivo educating the NK T cells obtained in step (a) such that the resulting educated NK T cells have the capability of modulating the Th1/Th2 cell balance toward anti-inflammatory cytokine producing cells; and
c. re-introducing to said subject the educated NK T cells obtained in step (b) which are capable of modulating the Th1/Th2 cell balance toward anti-inflammatory cytokine producing cells, resulting in an increase in the quantitative ratio between any one of IL4 and IL10 to IFNγ.

7. A method according to claim 6, wherein said ex vivo education of step (b) is performed by culturing said NK T cells in the presence of any one of:
a. antigens associated with said immune-related disorder or any combination thereof;
b. at least one of liver-associated cells of tolerized or non-tolerized subjects suffering from said immune-related disorder or of said subject;
c. at least one of cytokines, adhesion molecules or any combination thereof; and
d. a combination of any of (a), (b) and (c).

8. The method according to claim 7 wherein said ex vivo education is performed by culturing said NK T cells in the presence of antigens associated with said immune-related disorder.

9. The method according to claim 8, wherein said antigens are any of allogeneic antigens obtained from a donor subject suffering from said immune-related disorders, xenogenic antigens, autologous antigens and recombinantly prepared antigens and any combinations thereof.

10. The method according to claim 7, wherein said liver-associated cells are selected from the group consisting of Kupffer cells, Stellate cells, liver endothelial cells, liver-associated stem cells and any other liver-related lymphocytes.

11. The method according to claim 7, wherein said cytokines are selected from the group consisting of IL4, IL10, TGFβ, IFN□□ IL12, IL2, IL18 and IL15.

12. The method according to claim 7, wherein said adhesion molecules are selected from the group consisting of Integrins, Selectin and ICAM.

13. The method according to claim 6, wherein said educated NK T cells are re-introduced to said subject by adoptive transfer.

14. The method according to any one of claims 6 and 10, optionally further comprising the step of eliciting in said subject immune modulation of the immune-related disorder by administering to said subject components, cells, tissues and/or organs derived from any one of allogeneic donors suffering from said immune-related disorder, xenogeneic sources and autologous sources, and immunologically functional equivalents, or combinations thereof.

15. The method according to claim 14, wherein said components, cells, tissues or organs are administered orally.

16. The method according to any one of claims 1 to 15, wherein said immune-related disorder is an inflammatory bowel disease (IBD).

17. The method according to claim 16, wherein said disease is Crohn's disease.

18. A method according to any one of claims 1 to 15, wherein said immune-related disorder is a malignancy selected from the group consisting of melanomas, carcinomas, lymphomas and sarcomas.

19. A method according to any one of claims 16 to 18, wherein said mammalian subject is a human patient.

20. A method according to claim 19, wherein said NK T cells are NK T cells expressing the CD56 marker.

21. A therapeutic composition for the treatment of an immune-related disorder in a mammalian subject, which composition comprises as an effective ingredient ex vivo educated autologous NK T cells capable of modulating the Th1/Th2 cell balance toward anti-inflammatory cytokine producing cells, and optionally further comprising pharmaceutically acceptable carrier, diluent, excipient and/or additive.

22. A therapeutic composition of claim 21, wherein said educated autologous NK T cells mediate increase in the quantitative ratio between any one of IL4 and IL10 to IFNγ.

23. The therapeutic composition according to claim 22, wherein said educated autologous NK T cell is obtained by ex vivo culture in the presence of any one of:
a. antigens associated with said immune-related disorder or any combination thereof;
b. at least one of liver-associated cells of tolerized or non-tolerized patients suffering from said immune-related disorder or of said subject;
c. at least one of cytokines, or adhesion molecules; and
d. a combination of any of (a), (b), (c) above;.

24. The therapeutic composition according to claim 23, wherein said educated autologous NK T cell is obtained by ex vivo culture in the presence of antigens associated with said immune-related disorder.

25. The therapeutic composition according to claim 24, wherein said antigens is any one of allogeneic antigens from donors suffering from said immune-related disorder, xenogeneic antigens, autologous antigens from said subject and recombinantly prepared antigens or any combinations thereof.

26. The therapeutic composition according to claim 23, wherein said liver-associated cells are selected from the group consisting of Kupffer cells, Stellate cells, liver endothelial cells and any other liver-related lymphocytes.

27. The therapeutic composition according to claim 23, wherein said cytokines are selected from the group consisting of IL4, IL10, TGFβ, □IFNγ, IL12 and IL15.

28. The therapeutic composition according to claim 23, wherein said adhesion molecules are selected from the group consisting of Integrins, Selectin and ICAM.

29. A therapeutic composition according to any one of claims 21 to 28, wherein said immune-related disorder is an intestinal inflammatory disease.

30. The therapeutic composition according to claim 29, wherein said intestinal inflammatory disease is Crohn's disease.

31. The therapeutic composition according to any one of claims 21 to 28, wherein said immune-related disorder is a malignancy selected from the group consisting of melanomas, carcinomas, lymphomas and sarcomas.

32. Use of an educated autologous NK T cell, in the manufacture of a therapeutic composition for modulating the Th1/Th2 cell balance toward anti-inflammatory cytokine producing cells, in a mammalian subject suffering of a immune-related disorder.

33. Use of an educated autologous NK T cell, in the manufacture of a therapeutic composition for the treatment of immune-related disorder in a mammalian subject, which educated autologous NK T cells are capable of modulating the Th1/Th2 cell balance toward anti-inflammatory cytokine producing cells.

34. Use according to any one of claims 32 and 33, wherein said educated autologous NK T cells mediate an increase in the quantitative ratio between any one of IL4 and IL10 to IFNγ.

35. Use according to claim 34, in the manufacturing of a therapeutic composition according to any one of claims 21 to 28.

36. An ex vivo educated autologous NK T cell for use in the treatment of immune-related disorders in a mammalian subject in need of such treatment.

37. The educated NK T cell according to claim 36, wherein said educated NK T cell has been ex vivo cultured in the presence of any one of:
a. antigens associated with said immune-related disorder or any combination thereof;
b. at least one of liver-associated cells of tolerized or non-tolerized patients suffering from said immune-related disorder or of said subject;
c. at least one of cytokines, or adhesion molecules; and
d. a combination of any of (a), (b) and (c) above.

38. The educated NK T cell according to claim 37, wherein said antigens are any one of allogeneic antigens of donors suffering from said immune-related disorder, xenogeneic antigens, autologous antigens of said subject and recombinantly prepared antigens or any combinations thereof.

39. The educated NK T cell according to claim 37, wherein said liver-associated cells are selected from the group consisting of Kupffer cells, Stellate cells, liver endothelial cells and any other liver-related lymphocytes.

40. The educated NK T cell according to claim 37, wherein said cytokines are selected from the group consisting of IL4, IL10, TGFβ, IFNγ, IL12 and IL15.

41. The educated NK T cell according to claim 37, wherein said adhesion molecules are selected from the group consisting of Integrins, Selectin and ICAM.

42. The educated NK T cell according to claim 37, wherein said immune-related disorder is an intestinal inflammatory disease.

43. The educated NK T cell according to claim 42, wherein said intestinal inflammatory disease is Crohn's disease.

44. The educated NK T cell according to claim 37, wherein said immune-related disorder is a malignancy selected from the group consisting of melanomas, carcinomas, lymphomas and sarcomas.

45. Use of an ex vivo educated autologous NK T cell in the treatment of immune-related disorders in a mammalian subject in need of such treatment.

46. Use according to claim 45, wherein said educated autologous NK T cell is according to any one of claims 37 to 41.

47. A therapeutic composition for the treatment of immune related disorder, which composition comprises as an effective ingredient an antibody that specifically recognizes NK T cells.

48. The therapeutic composition according to claim 47, wherein said immune related disorder is an intestinal inflammatory disease.

49. The therapeutic composition according to claim 48, wherein said intestinal inflammatory disease is Crohn's disease.

50. The therapeutic composition according to claim 47, wherein said immune related disorder is a malignancy selected from the group consisting of melanomas, carcinomas, lymphomas and sarcomas.

51. Use of an antibody that specifically recognizes the NK T cells, in the manufacture of a therapeutic composition for manipulation of the NK T cells population in a mammalian subject suffering from an immune-related disorder.

52. The use according to claim 51, wherein said manipulation is depletion of said NK T cell population.

53. The use according to claim 52, wherein depletion of said NK T cells population results in modulating the Th1/Th2 cell balance toward anti-inflammatory cytokine producing cells.

54. The use according to an antibody that specifically recognizes NK T cells, in the manufacture of a therapeutic composition for the treatment of immune-related disorder in a mammalian subject.

55. The use according to any one of claims 51 to 54, wherein said immune related disorder is intestinal inflammatory disease.

56. The use according to claim 55, wherein said intestinal inflammatory disease is Crohn's disease.

57. The use according to any one of claims 51 to 56, wherein said immune-related disorder is a malignancy selected from the group consisting of melanomas, carcinomas, lymphomas and sarcomas.

58. A method for the treatment of immune-related disorders in a mammalian subject in need of such treatment, by manipulating NK T cell population of said subject, wherein manipulation of said NK T cell population results in modulation of the Th1/Th2 cell balance toward pro-inflammatory cytokine producing cells, said modulation being mediated by different components of said subject's immune system.

59. The method according to claim 58, wherein said components are selected from the group consisting of cellular immune reaction elements, humoral immune reaction elements and cytokines.

60. The method according to claim 58, wherein said manipulation is performed by depletion of said NKT cell population.

61. A method for treatment of immune-related disorders in a mammalian subject according to claim 58, comprising the steps of:
a. obtaining NK T cells from said subject;
b. ex vivo educating the NK T cells obtained in step (a) such that the resulting educated NK T cells have the capability of modulating the Th1/Th2 cell balance toward pro-inflammatory cytokine producing cells; and
c. re-introducing to said subject the educated NK T cells obtained in step (b) which are capable of modulating the Th1/Th2 cell balance toward pro-inflammatory cytokine producing cells, resulting in a decrease in the quantitative ratio between any one of IL4 and IL10 to IFNγ.

62. A method according to claim 61, wherein said ex vivo education of step (b) is performed by culturing said NK T cells in the presence of any one of:
a. antigens associated with said immune-related disorder or any combination thereof;
b. at least one of liver-associated cells of tolerized or non-tolerized subjects suffering from said immune-related disorder or of said subject;
c. at least one of cytokines, adhesion molecules or any combination thereof; and
d. a combination of any of (a), (b) and (c).

63. The method according to claim 62 wherein said ex vivo education is performed by culturing said NK T cells in the presence of antigens associated with said immune-related disorder.

64. The method according to claim 63, wherein said antigens are any of allogeneic antigens obtained from a donor subject suffering from said immune-related disorders, xenogenic antigens, autologous antigens and recombinantly prepared antigens and any combinations thereof.

65. The method according to claim 60, wherein said liver-associated cells are selected from the group consisting of Kupffer cells, Stellate cells, liver endothelial cells, liver-associated stem cells and any other liver-related lymphocytes.

66. The method according to claim 62, wherein said cytokines are selected from the group consisting of IL4, IL10, TGFβ, IFN□□ IL12, IL2, IL18 and IL15.

67. The method according to claim 62, wherein said adhesion molecules are selected from the group consisting of Integrins, Selectin and ICAM.

68. The method according to claim 61, wherein said educated NK T cells are re-introduced to said subject by adoptive transfer.

69. The method according to any one of claims 61 and 65, optionally further comprising the step of eliciting in said subject immune modulation of the immune-related disorder by administering to said subject components, cells, tissues and/or organs derived from any one of allogeneic donors suffering from said immune-related disorder, xenogeneic sources and autologous sources, and immunologically functional equivalents, or combinations thereof.

70. The method according to claim 69, wherein said components, cells, tissues or organs are administered orally.

71. A method according to any one of claims 58 to 70, wherein said immune-related disorder is a malignancy selected from the group consisting of melanomas, carcinomas, lymphomas and sarcomas.

72. A method according to claim 71, wherein said mammalian subject is a human patient.

73. A method according to claim 72, wherein said NK T cells are NK T cells expressing the CD56 marker.

74. A therapeutic composition for the treatment of an immune-related disorder in a mammalian subject, which composition comprises as an effective ingredient ex vivo educated autologous NK T cells capable of modulating the Th1/Th2 cell balance toward pro-inflammatory cytokine producing cells, and optionally further comprising pharmaceutically acceptable carrier, diluent, excipient and/or additive.

75. A therapeutic composition of claim 74, wherein said educated autologous NK T cells mediate a decrease in the quantitative ratio between any one of IL4 and IL10 to IFNγ.

76. The therapeutic composition according to claim 75, wherein said educated autologous NK T cell is obtained by ex vivo culture in the presence of any one of:
a. antigens associated with said immune-related disorder or any combination thereof;
b. at least one of liver-associated cells of tolerized or non-tolerized patients suffering from said immune-related disorder or of said subject;
c. at least one of cytokines, or adhesion molecules; and
d. a combination of any of (a), (b), (c) above;.

77. The therapeutic composition according to claim 76, wherein said educated autologous NK T cell is obtained by ex vivo culture in the presence of antigens associated with said immune-related disorder.

78. The therapeutic composition according to claim 77, wherein said antigens is any one of allogeneic antigens from donors suffering from said immune-related disorder, xenogeneic antigens, autologous antigens from said subject and recombinantly prepared antigens or any combinations thereof.

79. The therapeutic composition according to claim 76, wherein said liver-associated cells are selected from the group consisting of Kupffer cells, Stellate cells, liver endothelial cells and any other liver-related lymphocytes.

80. The therapeutic composition according to claim 76, wherein said cytokines are selected from the group consisting of IL4, IL10, TGFβ, □IFNγ, IL12 and IL15.

81. The therapeutic composition according to claim 76, wherein said adhesion molecules are selected from the group consisting of Integrins, Selectin and ICAM.

82. The therapeutic composition according to any one of claims 74 to 81, wherein said immune-related disorder is a malignancy selected from the group consisting of melanomas, carcinomas, lymphomas and sarcomas.

83. Use of an educated autologous NK T cell, in the manufacture of a therapeutic composition for modulating the Th1/Th2 cell balance toward pro-inflammatory cytokine producing cells, in a mammalian subject suffering of a immune-related disorder.

84. Use of an educated autologous NK T cell, in the manufacture of a therapeutic composition for the treatment of immune-related disorder in a mammalian subject, which educated autologous NK T cells are capable of modulating the Th1/Th2 cell balance toward pro-inflammatory cytokine producing cells.

85. Use according to any one of claims 83 and 85, wherein said educated autologous NK T cells mediate a decrease in the quantitative ratio between any one of IL4 and IL10 to IFNγ.

86. Use according to claim 85, in the manufacturing of a therapeutic composition according to any one of claims 74 to 81.

87. An ex vivo educated autologous NK T cell for use in the treatment of immune-related disorders in a mammalian subject in need of such treatment.

88. The educated NK T cell according to claim 87, wherein said educated NK T cell has been ex vivo cultured in the presence of any one of:
a. antigens associated with said immune-related disorder or any combination thereof;
b. at least one of liver-associated cells of tolerized or non-tolerized patients suffering from said immune-related disorder or of said subject;
c. at least one of cytokines, or adhesion molecules; and
d. a combination of any of (a), (b) and (c) above.

89. The educated NK T cell according to claim 88, wherein said antigens are any one of allogeneic antigens of donors suffering from said immune-related disorder, xenogeneic antigens, autologous antigens of said subject and recombinantly prepared antigens or any combinations thereof.

90. The educated NK T cell according to claim 88, wherein said liver-associated cells are selected from the group consisting of Kupffer cells, Stellate cells, liver endothelial cells and any other liver-related lymphocytes.

91. The educated NK T cell according to claim 88, wherein said cytokines are selected from the group consisting of IL4, IL10, TGFβ, IFNγ, IL12 and IL15.

92. The educated NK T cell according to claim 88, wherein said adhesion molecules are selected from the group consisting of Integrins, Selectin and ICAM.

93. The educated NK T cell according to claim 88, wherein said immune-related disorder is a malignancy selected from the group consisting of melanomas, carcinomas, lymphomas and sarcomas.

94. Use of an ex vivo educated autologous NK T cell in the treatment of immune-related disorders in a mammalian subject in need of such treatment.

95. Use according to claim 94, wherein said educated autologous NK T cell is according to any one of claims 88 to 92.

96. A therapeutic composition for the treatment of immune related disorder, which composition comprises as an effective ingredient an antibody that specifically recognizes NK T cells.

97. The therapeutic composition according to claim 96, wherein said immune related disorder is a malignancy selected from the group consisting of melanomas, carcinomas, lymphomas and sarcomas.

98. Use of an antibody that specifically recognizes the NK T cells, in the manufacture of a therapeutic composition for manipulation of the NK T cells population in a mammalian subject suffering from an immune-related disorder.

99. The use according to claim 98, wherein said manipulation is depletion of said NK T cell population.

100. The use according to claim 99, wherein depletion of said NK T cells population results in modulating the Th1/Th2 cell balance toward pro-inflammatory cytokine producing cells.

101. The use according to an antibody that specifically recognizes NK T cells, in the manufacture of a therapeutic composition for the treatment of immune-related disorder in a mammalian subject.

102. The use according to any one of claims 98 to 101, wherein said immune-related disorder is a malignancy selected from the group consisting of melanomas, carcinomas, lymphomas and sarcomas.
